(19) United States
(12) Patent Application Publication
(10) Pub. No.: US 2013/0065757 A1
(43) Pub. Date: Mar. 14, 2013

(54) CHEMICAL COMPOUNDS

(71) Applicants: William Guy Whittingham, Bracknell (GB); Shuji Hachisu, Bracknell (GB); Mary Bernadette Aspinall, Bracknell (GB); Matthew Brian Hotson, Bracknell (GB)

(72) Inventors: William Guy Whittingham, Bracknell (GB); Shuji Hachisu, Bracknell (GB); Mary Bernadette Aspinall, Bracknell (GB); Matthew Brian Hotson, Bracknell (GB)

(73) Assignee: Syngenta Limited, Bracknell, Berkshire (GB)

(21) Appl. No.: 13/698,578

(22) PCT Filed: May 16, 2011
(86) PCT No.: PCT/GB11/00748

§ 371 Date: Nov. 16, 2012

(30) Foreign Application Priority Data
May 18, 2010 (GB) 1008290.7

(51) Int. Cl. C07D 213/79; A01P 13/00; C07D 401/04; A01N 43/40; A01N 25/32

(52) U.S. Cl. 504/103; 546/310; 504/260; 546/276.4; 504/252

(57) ABSTRACT

The present invention relates to certain substituted picolinic acid derivatives, as well as N-oxides and agriculturally acceptable salts thereof, and their use in controlling plant growth, particularly undesirable plant growth, in crops of useful plants. The invention extends to herbicidal compositions comprising such compounds, N-oxides and/or salts as well as mixtures of the same with one or more further active ingredients and/or a safener.

The present invention relates to certain substituted picolinic acid derivatives, to processes for their preparation, herbicidal compositions comprising them and their use in controlling plants or inhibiting plant growth.

Herbicidal 4-aminopicolinates are disclosed in WO 01/51468, WO 03/011853, WO 2004/089906, WO 2005/016887 and WO 2006/062979.

In part, due to the evolution of herbicide-resistant weed populations, and herbicide-resistant crops becoming volunteer weeds, there is a continuing need to control such undesired plant growth in particular in crops of useful plants. Other factors, for example, the demand for cheaper, more effective herbicides, and for herbicides with an improved environmental profile (e.g. safer, less toxic etc.) also drive the need to identify novel herbicidal compounds.

It has now been found that certain picolinic acid derivatives display pre- and post-emergence herbicidal activity.

Accordingly, the present invention provides a compound of formula (I)

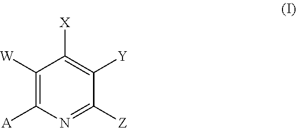

or salt or N-oxide thereof, wherein:

A is halogen, cyano, optionally substituted alkoxy, optionally substituted aryloxy, optionally substituted heteroaryloxy, optionally substituted alkylthio, optionally substituted arylthio, or optionally substituted heteroarylthio;

W is hydrogen, halogen, cyano, nitro, hydroxyl, amino, optionally substituted alkyl, optionally substituted haloalkyl, optionally substituted cycloalkyl, optionally substituted alkoxy, optionally substituted alkylamino, optionally substituted dialkylamino, optionally substituted alkylthio, optionally substituted alkylsulphinyl, optionally substituted alkylsulphonyl or optionally substituted aryl;

X is azido, nitro, optionally substituted alkoxy, optionally substituted alkylthio or —N R⁵R⁶ and
(i) R⁵ is hydrogen, optionally substituted C₁₋₄ alkyl provided said substitution does not comprise a ring system, optionally substituted C₁₋₄ haloalkyl provided said substitution does not comprise a ring system, optionally substituted C₃₋₆ cycloalkyl, C₂₋₄ alkenyl, C₂₋₄ alkynyl, —SO₂R², or —C(O)R³ and R⁶ is hydrogen, optionally substituted C₁₋₄ alkyl provided said substitution does not comprise a ring system, optionally substituted C₁₋₄ haloalkyl provided said substitution does not comprises a ring system, optionally substituted C₃₋₆ cycloalkyl, C₂₋₄ alkenyl, or C₂₋₄ alkynyl, or
(ii) R⁵ and R⁶ together form a group ═C(R⁸)OR⁹, ═C(R¹⁰)SR⁹, ═C(R¹¹)NR⁷₂, or
(iii) R⁵ and R⁶ together with the N atom to which they are attached form a 3 to 8 membered optionally substituted heterocyclyl or heteroaryl ring system, said ring system optionally containing 1 to 2 further heteroatoms independently selected from O, S and N,
   and R² is optionally substituted C₁₋₄ alkyl or phenyl optionally substituted by 1 to 3 groups R⁴, R³ is optionally substituted C₁₋₄ alkyl, phenyl optionally substituted by 1 to 3 groups R⁴, C₁₋₄ alkoxy, or —NR⁷₂, each R⁴ is independently halogen, C₁₋₄ alkyl, C₁₋₄ alkoxy, or C₁₋₄ alkylsulphonyl, R⁸ is hydrogen, C₁₋₄ alkyl, C₃₋₆ cycloalkyl, phenyl, C₁₋₄ alkoxy, C₁₋₄ alkylthio, or —NR⁷₂, R⁹ is C₁₋₄ alkyl, R¹⁰ is hydrogen, C₁₋₄ alkyl, C₃₋₆ cycloalkyl, phenyl, C₁₋₄ alkylthio, or —NR⁷₂, R¹¹ is hydrogen, C₁₋₄ alkyl, C₃₋₆ cycloalkyl, phenyl, or —NR⁷₂, and each R⁷ is independently hydrogen or C₁₋₄ alkyl;

Y is optionally substituted alkyl, optionally substituted haloalkyl, optionally substituted cycloalkyl, optionally substituted alkenyl, optionally substituted alkynyl or optionally substituted aryl;

Z is —C(O)R¹², —C(S)R¹³, or —C(═NR¹⁴)R¹⁵ and R¹² is hydrogen, hydroxyl, optionally substituted alkoxy, optionally substituted alkenyloxy, optionally substituted cycloalkoxy, optionally substituted alkylthio, amino, optionally substituted alkylamino or optionally substituted dialkylamino, R¹³ is optionally substituted alkoxy, optionally substituted cycloalkoxy, optionally substituted alkylthio, amino, optionally substituted alkylamino or optionally substituted dialkylamino, R¹⁴ is hydrogen, optionally substituted alkyl, optionally substituted alkoxy, optionally substituted cycloalkoxy, amino, optionally substituted alkylamino or optionally substituted dialkylamino and R¹⁵ is hydrogen, optionally substituted alkoxy, optionally substituted cycloalkoxy, optionally substituted alkylthio, amino, optionally substituted alkylamino or optionally substituted dialkyamino.

“Alkyl” means a linear saturated monovalent hydrocarbon radical of one to twenty carbon atoms or a branched saturated monovalent hydrocarbon radical of three to twenty carbon atoms, e.g. methyl, ethyl, n-propyl, iso-propyl, n-butyl, sec-butyl, iso-butyl, tert-butyl, and the like. Suitably, linear alkyl groups contain one to ten, one to six, one to five or one to four carbon atoms, more suitably are selected from methyl, ethyl or n-propyl and, most suitably, are methyl or ethyl. Suitably, branched alkyl groups contain three to ten, three to six or three to five carbon atoms and more suitably are selected from iso-propyl, sec-butyl, iso-butyl or tert-butyl. It is noted that this definition applies both when the term is used alone and when it is used as part of a compound term, such as “haloalkyl” and similar terms.

“Cycloalkyl” means a monovalent cyclic hydrocarbon radical of three to ten ring carbons e.g. cyclopropyl, cyclobutyl, cyclopentyl and cyclohexyl. Cycloalkyl groups are fully saturated. Suitably, cycloalkyl groups contain three to six carbon atoms and, more suitably, are cyclopropyl or cyclobutyl.

“Alkenyl” means a linear monovalent unsaturated hydrocarbon radical of two to ten carbon atoms, or a branched monovalent hydrocarbon radical of three to ten carbon atoms containing at least one double bond, e.g. ethenyl, propenyl and the like. Where appropriate, an alkenyl group can be of either the (E)- or (Z)-configuration. Suitably, linear alkenyl groups contain two to six carbon atoms, more suitably two to four carbon atoms and, most suitably are ethenyl (vinyl), prop-1-enyl (1-propenyl) or prop-2-enyl (allyl). Suitably, branched alkenyl groups contain three to six carbon atoms, more suitably from three to four and, most suitably, are 1-methylethenyl (2-propenyl), 1-methylprop-1-enyl, 1-methylprop-2-enyl, 2-methylprop-1-enyl and 2-methylprop-2-enyl (2-methylallyl).

“Alkynyl” means a linear monovalent unsaturated hydrocarbon radical of two to ten carbon atoms, e.g. ethynyl, propynyl and the like. Suitably, alkynyl groups contain two to six carbon atoms and more suitably two to four carbon atoms e.g. ethynyl, prop-1-ynyl, prop-2-ynyl, but-1-ynyl, but-2-ynyl and but-3-ynyl.

“Alkoxy” means a radical —OR, where R is alkyl as defined above. Alkoxy groups include, but are not limited to, methoxy, ethoxy, 1-methylethoxy, propoxy, butoxy, 1-methylpropoxy and 2-methylpropoxy. Preferably alkoxy means methoxy or ethoxy.

“Cycloalkoxy” means a radical —OR, where R is cycloalkyl as defined above.

“Alkoxyalkyl” means a radical —ROR, where each R is, independently, alkyl as defined above.

“Alkoxyalkoxy” means a radical —OROR, wherein each R is, independently, alkyl as defined above.

“Aryl” or “aromatic ring” refers to an aromatic substituent which may be a single ring or multiple rings which are fused together, linked covalently or linked to a common group such as an ethylene (—CH₂—CH₂—) or methylene (—CH₂—) moiety. Representative examples of aryl include, for example, phenyl, naphthyl, azulenyl, indanyl, indenyl, anthracenyl, phenanthrenyl, tetrahydronaphthyl, biphenyl, diphenylmethyl and 2,2-diphenyl-1-ethyl. Preferred aryl groups are phenyl and naphthyl groups.

“Heteroaryl” means a ring system consisting either of a single aromatic ring or of two or more fused rings, at least one of which is aromatic, the other or others independently being saturated, unsaturated or aromatic, containing one, two, three or four ring heteroatoms selected, independently, from N, O or S, the remaining ring atoms being carbon. Examples of heteroaryl groups include, but are not limited to pyridyl, pyridazinyl, pyrimidinyl, pyrazinyl, triazinyl, furanyl, thiophenyl, oxazolyl, isoxazolyl, oxadiazolyl, thiazolyl, isothiazolyl, thiadiazolyl, pyrrolyl, pyrazolyl, imidazolyl, triazolyl and tetrazolyl. Examples of bicyclic groups are benzothiophenyl, benzimidazolyl, benzothiadiazolyl, quinolinyl, cinnolinyl, quinoxalinyl and pyrazolo[1,5-a]pyrimidinyl. Preferred heteroaryl groups include pyridyl, pyrimidinyl, furanyl, thiophenyl, thiazolyl, pyrrolyl, pyrazolyl, imidazolyl, triazolyl, benzothiophenyl, benzimidazolyl and quinolinyl.

“Heterocyclyl” means a ring system consisting either of a single ring or of two or more fused rings, which may be saturated or unsaturated, containing one, two, three or four ring heteroatoms selected, independently, from N, O or S, the remaining atoms being carbon. Examples of heterocyclyl groups include, but are not limited to pyrrolidinyl, imidazolinyl, pyrazolidinyl, piperidyl, piperazinyl, quinuclidinyl, morpholinyl, together with unsaturated or partially unsaturated analogues such as 4,5,6,7-tetrahydro-benzothiophenyl, chromen-4-onyl, 9H-fluorenyl, 3,4-dihydro-2H-benzo-1,4-dioxepinyl, 2,3-dihydro-benzofuranyl, piperidinyl, 1,3-dioxolanyl, 1,3-dioxanyl, 4,5-dihydro-isoxazolyl, tetrahydrofuranyl and morpholinyl. Preferred heterocyclyl groups include aziridinyl, azetidinyl, pyrrolidinyl, piperidinyl, piperazinyl and morpholinyl.

“Halo” or “halogen” means fluoro, chloro, bromo or iodo, preferably chloro or fluoro.

“Haloalkyl” means alkyl as defined above substituted with one or more of the same or different halo atoms. Examples of haloalkyl groups include, but are not limited to chloromethyl, fluoromethyl, dichloromethyl, difluoromethyl, trichloromethyl, trifluoromethyl, 2-fluoroethyl, 2,2,2-trifluoroethyl, 1,1-difluoroethyl, 1,2-difluoroethyl, 2,2-difluoroethyl, pentafluoroethyl, 2-chloroethyl, 3-fluoropropyl, 3-chloropropyl, 2,2,2-trifluoro-1-chloroethyl and heptafluoropropyl.

“Haloalkenyl” means alkenyl as defined above substituted with one or more of the same or different halo atoms. Examples of haloalkenyl groups include, but are not limited to 2,2-dibromoethenyl, 2-fluoro-2-bromoethenyl, and 3,3-dichloroprop-2-enyl.

“Haloalkoxy” means a radical —OR, wherein R is haloalkyl as defined above.

“Aryloxy” means a radical —OR, wherein R is an aryl group as defined above.

“Heteroaryloxy” means a radical —OR, wherein R is a heteroaryl group as defined above.

“Alkylthio” means a radical —SR, where R is an alkyl group as defined above. Alkylthio groups include, but are not limited to, methylthio, ethylthio, propylthio, tert-butylthio, and the like.

“Haloalkylthio” means a radical —SR, where R is a haloalkyl group as defined above.

“Arylthio” means a radical —SR, wherein R is an aryl group as defined above.

“Heteroarylthio” means a radical —SR, wherein R is a heteroaryl group as defined above.

“Alkylsulphinyl” means —S(O)R, wherein R is an alkyl group as defined above.

“Alkylsulphonyl” means —S(O)₂R, wherein R is an alkyl group as defined above.

“Alkylsulphonylamino” means a radical —NHS(O)₂R, wherein R is an alkyl group as defined above.

“Alkylcarbonyl” means a radical —C(O)R, wherein R is alkyl as defined above.

“Aminocarbonyl” means a radical —C(O)NH₂.

“Alkylcarbonylamino” means a radical —NHC(O)R, wherein R is alkyl as defined above

“Cyano” means a —CN group.

“Hydroxy” or “hydroxyl” means an —OH group.

“Nitro” means an —NO₂ group.

“Amino” means an —NH₂ group.

“Carboxy” or “carboxyl” means a —C(O)OH group.

“Azido” means the group —N═N═N.

“Oxo” means the group ═O.

“Alkylamino” means a radical —NRH, where R is alkyl as defined above. Examples of alkylamino groups are methylamino, ethylamino, n-propylamino, i-propylamino etc “Dialkylamino” means a radical —NRR, where each R is, independently, alkyl as defined above. Examples of dialkylamino groups are dimethylamino, diethylamino, di-n-propylamino, methylethylamino, methylsopropylamino, etc.

“Alkoxycarbonyl” means —C(O)OR, wherein R is an alkyl group as defined above. Examples of alkoxycarbonyl groups include methoxycarbonyl, ethoxycarbonyl, i-propoxycarbonyl, n-propoxycarbonyl, n-butoxycarbonyl and s-butoxycarbonyl etc.

“Alkylcarbonyloxy” means —OC(O)R, wherein R is an alkyl group as defined above.

“Arylalkoxy” means —OR-aryl, wherein R is an alkyl group as defined above.

“Trialkylsilyl” means the group —Si(R)₃, wherein each R is, independently, an alkyl group as defined above.

The groups defined above when used alone or as part of a compound term (e.g. alkyl when used alone or as part of, for example, haloalkyl) may be optionally substituted where possible by one or more substituents, preferably by one, two, three or four substituents. In particular, alkyl, haloalkyl, alkenyl, alkynyl, cycloalkyl, alkoxy, cycloalkoxy, haloalkoxy, alkylamino, dialkylamino, alkylthio, haloalkylthio, alkylsulphinyl, alkylsulphonyl, aryl, heteroaryl, aryloxy, arylthio, heteroaryloxy and heteroarylthio groups may be optionally substituted.

Suitably, for alkyl, haloalkyl, alkenyl, alkynyl, alkoxy, haloalkoxy, alkylamino, dialkylamino, alkylthio, haloalkylthio, alkylsulphinyl, alkylsulphonyl, cycloalkyl or cycloalkoxy groups, these optional substituents are independently selected from hydroxyl, halogen, cyano, nitro, alkyl, haloalkyl, cycloalkyl, alkoxy, haloalkoxy, alkoxyalkoxy, alkylcarbonyloxy, carboxyl, trialkylsilyl, aryl (in particular, phenyl), heteroaryl, amino, alkylamino, dialkylamino, —SR^{a}, —S(O)R^{a}, S(O)₂R^{a} (wherein R^{a} is alkyl, haloalkyl, alkenyl, alkynyl, cycloalkyl or alkylcarbonylamino), C(O)R^{b} (wherein R^{b} is hydrogen, hydroxyl, alkyl, haloalkyl, cycloalkyl, alkoxy, arylalkoxy (in particular phenylalkoxy), cycloalkoxy, amino, alkylamino, dialkylamino or alkylsulphonylamino) or any two geminal optional substituents may form an oxo group. Aryl and heteroaryl optional substituents on these groups may be further substituted by one or more substituents independently selected from halogen, cyano, alkyl, haloalkyl, alkoxy, haloalkoxy, alkoxyalkyl, alkylsulphonyl or alkoxycarbonyl.

Suitably, for aromatic groups, these optional substituents are independently selected from hydroxyl, halogen, cyano, nitro, alkyl, haloalkyl, cycloalkyl, alkoxy, haloalkoxy, alkoxyalkoxy, alkylcarbonyloxy, amino, alkylamino, dialkylamino, —SR^{a}, —S(O)R^{a}, S(O)₂R^{a} (wherein R^{a} is alkyl, haloalkyl, alkenyl, alkynyl, cycloalkyl or alkylcarbonylamino), C(O)R^{b} (wherein R^{b} is hydrogen, hydroxyl, alkyl, haloalkyl, cycloalkyl, alkoxy, arylalkoxy (in particular phenylalkoxy), cycloalkoxy, amino, alkylamino, dialkylamino or alkylsulphonylamino) or any two geminal optional substituents may form, where possible, a group selected from oxo, ═CR^{d}₂, ═NOR^{e} or ═NNR^{e}R^{f} (wherein each R^{d} is, independently, hydrogen, halogen, cyano, nitro, alkyl, cycloalkyl, alkylcarbonyl, alkoxycarbonyl, alkylsulphonyl or aminocarbonyl; each R^{e} is, independently, hydrogen, alkyl or cycloalkyl; each R^{f} is, independently, hydrogen or alkyl). Optional substituents on an aromatic ring may be further substituted by one or more substituents independently selected from hydroxyl, cyano, cycloalkyl, alkoxy, haloalkoxy, alkoxyalkoxy, alkylcarbonyloxy, amino, alkylamino, dialkylamino, —SR^{a}, —S(O)R^{a}, S(O)₂R^{a} (wherein R^{a} is alkyl, haloalkyl, alkenyl, alkynyl, cycloalkyl or alkylcarbonylamino), C(O)R^{b} (wherein R^{b} is hydrogen, hydroxyl, alkyl, haloalkyl, cycloalkyl, alkoxy, arylalkoxy (in particular phenylalkoxy), cycloalkoxy, amino, alkylamino, dialkylamino or alkylsulphonylamino).

It is noted that, when X is —NR⁵R⁶ and either or both of R⁵ and R⁶ are optionally substituted alkyl or haloalkyl, said substitution does not comprise a ring system. In this context, a ‘ring system’ encompasses cycloalkyl, aryl, heteroaryl and heterocyclyl ring systems and substitutions wherein said ring systems form part of a compound substitution for example, aryloxy and arylalkoxy.

The compounds of formula I may exist in different geometric or optical isomeric forms or in different tautomeric forms. One or more centres of chirality may be present, in which case compounds of the formula I may be present as pure enantiomers, mixtures of enantiomers, pure diastereomers or mixtures of diastereomers. There may be double bonds present in the molecule, such as C═C or C═N bonds, in which case compounds of formula I may exist as single isomers or mixtures of isomers. Centres of tautomerisation may be present. This invention covers all such isomers and tautomers and mixtures thereof in all proportions as well as isotopic forms such as deuterated compounds.

Suitable salts include those formed by contact with acids or bases. Suitable salts of the compounds of formula I thus include those derived from alkali or alkaline earth metals and those derived from ammonia and amines. Preferred cations include sodium, potassium, magnesium, and ammonium cations of the formula N⁺(RⁱR^{j}R^{k}R^{l}) wherein Rⁱ, R^{j}, R^{k} and R^{l} are independently selected from hydrogen, C₁₋₆ alkyl and C₁₋₆ hydroxyalkyl. Salts of the compounds of formula I can be prepared by treatment of compounds of formula I with a metal hydroxide, such as sodium hydroxide, or an amine, such as ammonia, trimethylamine, diethanolamine, 2-methylthiopropylamine, bisallylamine, 2-butoxyethylamine, morpholine, cyclododecylamine, or benzylamine. Amine salts are often preferred forms of the compounds of Formula I because they are water-soluble and lend themselves to the preparation of desirable aqueous based herbicidal compositions.

Suitable salts of the compounds of formula I also include acid addition salts such as those with an inorganic acid such as hydrochloric, hydrobromic, sulphuric, nitric or phosphoric acid, an organic acid such as acetic, butyric, propionic or hexanoic, an organic carboxylic acid such as citric, fumaric, lactic, maleic, malonic, mandelic, malic, oxalic, succinic, tartaric, toluic or phthalic acid, or a sulphonic acid such as methane, benzene, naphthalene, camphor or toluene sulphonic acid.

N-oxides are oxidised forms of tertiary amines or oxidised forms of nitrogen containing heteroaromatic compounds. They are described in many books for example in “Heterocyclic N-oxides” by Angelo Albini and Silvio Pietra, CRC Press, Boca Raton, Fla., 1991.

In particularly preferred embodiments of the invention, the preferred groups for A, W, X, Y and Z, in any combination thereof, are as set out below.

In a preferred embodiment, A is halogen, cyano, C₁₋₆ alkoxy optionally substituted by 1 to 3 groups R¹⁶, C₁₋₆ haloalkoxy optionally substituted by 1 to 3 groups R¹⁶, C₁₋₆ alkylthio optionally substituted by 1 to 3 groups R¹⁶, C₁₋₆ haloalkylthio optionally substituted by 1 to 3 groups R¹⁶, aryloxy optionally substituted by 1 to 3 groups R¹, heteroaryloxy optionally substituted by 1 to 3 groups R¹, arylthio optionally substituted by 1 to 3 groups R¹ or heteroarylthio optionally substituted by 1 to 3 groups R¹, each R¹⁶ is independently cyano, hydroxyl, C₃₋₆ cycloalkyl, —OR¹⁷, —S(O)ₐR¹⁸, —C(O)R¹⁹ or —NR²⁰₂, each R¹ is independently halogen, cyano, nitro, hydroxyl, C₁₋₆ alkyl optionally substituted by 1 to 4 groups R¹⁶, C₁₋₆ haloalkyl optionally substituted by 1 to 4 groups R¹⁶, —OR¹⁷, —S(O)ₐR¹⁸, —C(O)R¹⁹, or —NR²⁰₂ or any two geminal groups R¹ together form a group selected from oxo, ═CR²¹₂, ═NOR²², or ═NNR²²R²³ and
(i) each R¹⁷ is independently C₁₋₆ alkyl, C₁₋₆ haloalkyl, C₁₋₄ alkoxy(C₁₋₄)alkyl, or C₁₋₆ alkylcarbonyl,
(ii) each R¹⁸ is independently C₁₋₆ alkyl, C₁₋₆ haloalkyl, C₂₋₆ alkenyl, C₂₋₆ alkynyl, C₃₋₆ cycloalkyl, or C₁₋₆ alkylcarbonylamino and each a is, independently, 0, 1, or 2,
(iii) each R¹⁹ is independently hydrogen, hydroxyl, C₁₋₆ alkyl, C₁₋₆ haloalkyl, C₃₋₆ cycloalkyl, C₁₋₆ alkoxy, phenyl(C₁₋₆)alkoxy, C₃₋₆ cycloalkoxy, amino, C₁₋₆ alkylamino, di(C₁₋₄)alkylamino, or C₁₋₆ alkylsulphonylamino,
(iv) each R²⁰ is independently hydrogen or C₁₋₄ alkyl,
(v) each R²¹ is independently hydrogen, halogen, cyano, nitro, C₁₋₆ alkyl, C₃₋₆ cycloalkyl, C₁₋₆ alkylcarbonyl, C₁₋₆ alkoxycarbonyl, C₁₋₆ alkylsulphonyl, or aminocarbonyl,
(vi) each R²² is independently hydrogen, C₁₋₆ alkyl, or C₃₋₆ cycloalkyl and
(vii) each R²³ is independently hydrogen or C₁₋₆ alkyl.

In a more preferred embodiment, A is halogen, C₁₋₄ alkylthio optionally substituted by 1 to 3 groups R¹⁶, C₁₋₄ haloalkylthio optionally substituted by 1 to 3 groups R¹⁶ or aryloxy optionally substituted by 1 to 3 groups R¹ wherein each R¹ and each R¹⁶ are, independently as defined above. In a yet more preferred embodiment, A is halogen or aryloxy optionally substituted by 1 to 3 groups R¹ wherein each R¹ is as defined above or, in a more preferred embodiment, each R¹ is independently halogen, cyano, C₁₋₂ alkyl, C₁₋₂ haloalkyl, C₁₋₂ alkoxy, C₁₋₂ haloalkoxy, or di(C₁₋₂)alkylamino. In a yet more preferred embodiment, A is halogen and, most preferably, A is chlorine.

In a preferred embodiment, W is hydrogen, halogen, cyano, nitro, hydroxyl, amino, C₁₋₆ alkyl optionally substituted by 1 to 3 groups R³⁰, C₁₋₆ haloalkyl optionally substituted by 1 to 3 groups R³⁰, C₃₋₆ cycloalkyl optionally substituted by 1 to 3 groups R³⁰, C₁₋₆ alkoxy optionally substituted by 1 to 3 groups R³⁰, C₁₋₆ alkylamino optionally substituted by 1 to 3 groups R³⁰, di(C₁₋₆)alkylamino optionally substituted by 1 to 3 groups R³⁰, C₁₋₆ alkylthio optionally substituted by 1 to 3 groups R³⁰, C₁₋₆ alkylsulphinyl optionally substituted by 1 to 3 groups R³⁰, C₁₋₆ alkylsulphonyl optionally substituted by 1 to 3 groups R³⁰ or C₅₋₁₀ aryl optionally substituted by 1 to 3 groups R³⁰ and each R³⁰ is independently selected from halogen, hydroxyl, cyano, amino, nitro, C₁₋₆ alkylamino, di(C₁₋₆)alkylamino, C₁₋₆ alkyl, C₁₋₆ haloalkyl, C₃₋₆ cycloalkyl, C₁₋₆ alkoxy, C₁₋₆ haloalkoxy, C₁₋₆ alkylthio, C₁₋₆ alkylcarbonyl or C₁₋₆ alkoxycarbonyl. In a yet more preferred embodiment, W is hydrogen, halogen, C₁₋₃ alkyl, C₁₋₃ haloalkyl, C₁₋₂ alkoxy(C₁₋₂)alkyl or cyclopropyl optionally substituted by 1 or 2 groups independently selected from halogen or C₁₋₆ alkyl. In a yet more preferred embodiment, W is hydrogen, halogen, C₁₋₂ alkyl, C₁₋₂ haloalkyl, C₁₋₂ alkoxy(C₁₋₂)alkyl, or cyclopropyl. In a yet more preferred embodiment, W is hydrogen or halogen and, most preferably, W is hydrogen, fluorine or chlorine.

In a preferred embodiment, X is azido, nitro, alkoxy optionally substituted by 1 to 3 groups R³¹, alkylthio optionally substituted by 1 to 3 groups R³¹ or —NR⁵R⁶, and
(i) R⁵ is hydrogen, C₁₋₄ alkyl optionally substituted by 1 to 4 groups R²⁴, C₁₋₄ haloalkyl optionally substituted by 1 to 4 groups R²⁴, C₃₋₆ cycloalkyl optionally substituted by 1 to 4 groups R²⁴, C₂₋₄ alkenyl, C₂₋₄ alkynyl, —SO₂R², or —C(O)R³ and R⁶ is hydrogen, C₁₋₄ alkyl optionally substituted by 1 to 4 groups R²⁴, C₁₋₄ haloalkyl optionally substituted by 1 to 4 groups R²⁴, C₃₋₆ cycloalkyl optionally substituted by 1 to 4 groups R²⁴, C₂₋₄ alkenyl, or C₂₋₄ alkynyl, or
(ii) R⁵ and R⁶ together form a group ═C(R⁸)OR⁹, ═C(R¹⁰)SR⁹, ═C(R¹¹)NR⁷₂ or
(iii) R⁵ and R⁶ together with the N atom to which they are attached form a 3 to 8 membered heterocyclyl or heteroaryl ring system, said ring system optionally containing 1 to 2 further heteroatoms independently selected from O, S and N and being optionally substituted by 1 to 3 groups R³³
   and R², R³, R⁸, R⁹, R¹⁰, R¹¹ and R⁷ are as defined above, each R³¹ is independently selected from halogen, hydroxyl, cyano, amino, nitro, C₁₋₆ alkylamino, di(C₁₋₆)alkylamino, C₃₋₆ cycloalkyl, aryl optionally substituted by 1 to 3 groups R³², heteroaryl optionally substituted by 1 to 3 groups R³², C₁₋₆ alkoxy, C₁₋₆ haloalkoxy, C₁₋₆ alkylthio, C₁₋₆ alkylcarbonyl or C₁₋₆ alkoxycarbonyl, each R²⁴ is independently halogen, hydroxyl, cyano, amino, nitro, C₁₋₆ alkylamino, di(C₁₋₆)alkylamino, C₁₋₆ alkyl, C₁₋₆ haloalkyl, C₁₋₆ alkoxy, C₁₋₆ haloalkoxy, C₁₋₆ alkoxy(C₁₋₆)alkoxy, carboxy, C₁₋₆ alkylthio, C₁₋₆ alkylcarbonyl C₁₋₆ alkoxycarbonyl or tri(C1-4)alkylsilyl, each R³² is independently selected from halogen, hydroxyl, cyano, amino, nitro, C₁₋₆ alkylamino, di(C₁₋₆)alkylamino, C₁₋₆ alkyl, C₁₋₆ haloalkyl, C₃₋₆ cycloalkyl, C₁₋₆ alkoxy, C₁₋₆ haloalkoxy, C₁₋₆ alkylthio, C₁₋₆ alkylcarbonyl or C₁₋₆ alkoxycarbonyl and each R³³ is independently halogen, hydroxyl, cyano, amino, nitro, C₁₋₆ alkylamino, di(C₁₋₆)alkylamino, C₁₋₆ alkyl, C₁₋₆ haloalkyl, C₁₋₆ alkoxy, C₁₋₆ haloalkoxy, C₁₋₆ alkylthio, C₁₋₆ alkylcarbonyl or C₁₋₆ alkoxycarbonyl or two geminal groups R³³ form an oxo group. Preferably, R² is C₁₋₄ alkyl optionally substituted by 1 to 4 groups R²⁵, C₁₋₄ haloalkyl optionally substituted by 1 to 4 groups R²⁵ or phenyl optionally substituted by 1 to 3 groups R⁴ wherein each R²⁵ is independently cyano, C₁₋₄ alkoxy, C₃₋₆ cycloalkyl, phenyl optionally substituted by 1-3 groups R⁴, heteroaryl optionally substituted by 1-3 groups R⁴, or C₁₋₄ alkoxycarbonyl and R⁴ is as defined above and, more preferably, R² is C₁₋₄ alkyl, C₁₋₄ haloalkyl or phenyl optionally substituted by 1 to 3 groups R⁴. Preferably, R³ is C₁₋₄ alkyl optionally substituted by 1 to 4 groups R²⁵, C₁₋₄ haloalkyl optionally substituted by 1 to 4 groups R²⁵, phenyl optionally substituted by 1 to 3 groups R⁴, C₁₋₄ alkoxy, or —NR⁷₂, wherein each R²⁵ is independently cyano, C₁₋₄ alkoxy, C₃₋₆ cycloalkyl, phenyl optionally substituted by 1-3 groups R⁴, heteroaryl optionally substituted by 1-3 groups R⁴, or C₁₋₄ alkoxycarbonyl and R⁴ and R⁷ are as defined above.

In a yet more preferred embodiment, X is —NR⁵R⁶ wherein R⁵ is hydrogen, C₁₋₄ alkyl optionally substituted by 1 to 4 groups R²⁴, C₁₋₄ haloalkyl optionally substituted by 1 to 4 groups R²⁴, C₃₋₆ cycloalkyl optionally substituted by 1 to 4 groups R²⁴, C₂₋₄ alkenyl, —SO₂R² or —C(O)R³ and R⁶ is hydrogen, C₁₋₄ alkyl optionally substituted by 1 to 4 groups R²⁴, C₁₋₄ haloalkyl optionally substituted by 1 to 4 groups R²⁴ or C₂₋₄ alkenyl or R⁵ and R⁶ together form a group ═C(R¹¹)NR⁷₂ and R² is C₁₋₄ alkyl, C₁₋₄ haloalkyl, or phenyl optionally substituted by 1 to 3 groups R⁴, R³ is C₁₋₄ alkyl optionally substituted by 1 to 4 groups R²⁵, C₁₋₄ haloalkyl optionally substituted by 1 to 4 groups R²⁵, phenyl optionally substituted by 1 to 3 groups R⁴, C₁₋₄ alkoxy, or —NR⁷₂, R¹¹ is hydrogen or C₁₋₄ alkyl, each R²⁴ is independently hydroxyl, cyano, C₁₋₄ alkoxy, C₁₋₄ alkoxy(C₁₋₄) alkoxy, carboxy, C₁₋₄ alkoxycarbonyl, or tri(C₁₋₄)alkylsilyl and R⁴, R⁷ and R²⁵ are as defined above.

In a yet more preferred embodiment, X is —NR⁵R⁶ wherein R⁵ is hydrogen, C₁₋₄ alkyl optionally substituted with 1 or 2 hydroxy or C₁₋₄ alkoxy groups, C₁₋₄ haloalkyl optionally substituted with 1 or 2 hydroxy or C₁₋₄ alkoxy groups, C₃₋₆ cycloalkyl, C₂₋₄ alkenyl, —SO₂R², or —C(O)R³, wherein R² and R³ are each independently C₁₋₃ alkyl or phenyl and R⁶ is hydrogen, C₁₋₄ alkyl optionally substituted with 1 or 2 hydroxy or C₁₋₄ alkoxy groups, C₁₋₄ haloalkyl optionally substituted with 1 or 2 hydroxy or C₁₋₄ alkoxy groups, or C₂₋₄ alkenyl. In a yet more preferred embodiment, X is —NR⁵R⁶ wherein R⁵ and R⁶ are, independently, hydrogen or C₁₋₄ alkyl and, most preferably, X is —NH₂.

In a preferred embodiment, Y is optionally substituted alkyl, optionally substituted haloalkyl, optionally substituted cycloalkyl, optionally substituted alkenyl or optionally substituted alkynyl. In a yet more preferred embodiment, Y is C₁₋₆ alkyl optionally substituted by 1 to 3 groups R³⁴, C₁₋₆ haloalkyl optionally substituted by 1 to 3 groups R³⁴, C₃₋₆ cycloalkyl optionally substituted by 1 to 3 groups R³⁵, C₂₋₆ alkenyl optionally substituted by 1 to 3 groups R³⁶ or C₂₋₆ alkynyl optionally substituted by 1 to 3 groups R³⁷ and each R³⁴ is independently halogen, cyano, nitro, hydroxyl, C₃₋₆ cycloalkyl, C₁₋₆ alkoxy, C₁₋₄ alkylthio, C₁₋₄ alkylcarbonyl, C₁₋₄ alkoxycarbonyl or two geminal groups R³⁴ form an oxo group, each R³⁵ is independently halogen, cyano, nitro, hydroxyl, C₁₋₆ alkyl, C₃₋₆ cycloalkyl, C₁₋₆ alkoxy, C₁₋₄ alkylthio, C₁₋₄ alkylcarbonyl or C₁₋₄ alkoxycarbonyl, each R³⁶ is independently halogen, cyano, nitro, C₃₋₆ cycloalkyl, C₁₋₆ alkoxy, C₁₋₄ alkylcarbonyl, alkoxycarbonyl or C₁₋₃alkylsulphonyl and each R³⁷ is independently halogen, cyano, C₃₋₆ cycloalkyl, C₁₋₄ alkylcarbonyl, C₁₋₄ alkoxycarbonyl or tri(C₁₋₃)alkylsilyl.

In a yet more preferred embodiment, Y is C₁₋₃ alkyl, C₁₋₃ haloalkyl, C₁₋₂ alkoxy(C₁₋₂)alkyl, cyclopropyl optionally substituted by 1 or 2 groups independently selected from halogen or C₁₋₆ alkyl, C₂₋₄ alkenyl, C₂₋₄ haloalkenyl or C₂₋₄ alkynyl optionally substituted by 1 or 2 groups independently selected from halogen or tri(C₁₋₃)alkylsilyl. In a yet more preferred embodiment, Y is C₁₋₂ alkyl, C₁₋₂ haloalkyl, C₁₋₂ alkoxy(C₁₋₂)alkyl, C₂₋₄ alkenyl or C₂₋₄ alkynyl. In a yet more preferred embodiment, Y is C₂₋₃ alkenyl and, most preferably, Y is ethenyl.

In a preferred embodiment, Z is —C(O)R¹², —C(S)R¹³, or —C(═NR¹⁴)R¹⁵ and R¹² is hydrogen, hydroxyl, optionally substituted alkoxy, optionally substituted cycloalkoxy, optionally substituted alkylthio, amino, optionally substituted alkylamino or optionally substituted dialkylamino, R¹³ is optionally substituted alkoxy, optionally substituted cycloalkoxy, optionally substituted alkylthio, amino, optionally substituted alkylamino or optionally substituted dialkylamino, R¹⁴ is hydrogen, optionally substituted alkyl, optionally substituted alkoxy, optionally substituted cycloalkoxy, amino, optionally substituted alkylamino or optionally substituted dialkylamino and R¹⁵ is hydrogen, optionally substituted alkoxy, optionally substituted cycloalkoxy, optionally substituted alkylthio, amino, optionally substituted alkylamino or optionally substituted dialkyamino.

In a more preferred embodiment, Z is —C(O)R¹², —C(S)R¹³, or —C(═NR¹⁴)R¹⁵ and R¹² is hydrogen, hydroxyl, C₁₋₂₀ alkoxy optionally substituted by 1 to 3 groups R³⁸, C₁₋₁₀ alkenyloxy optionally substituted by 1 to 3 groups R³⁸, C₃₋₆ cycloalkoxy optionally substituted by 1 to 3 groups R³⁸, C₁₋₁₀ alkylthio optionally substituted by 1 to 3 groups R³⁸, amino, C₁₋₆ alkylamino optionally substituted by 1 to 3 groups R³⁸ or di(C₁₋₆)alkylamino optionally substituted by 1 to 3 groups R³⁸, R¹³ is C₁₋₂₀ alkoxy optionally substituted by 1 to 3 groups R³⁸, C₃₋₆ cycloalkoxy optionally substituted by 1 to 3 groups R³⁸, C₁₋₁₀ alkylthio optionally substituted by 1 to 3 groups R³⁸, amino, C₁₋₆ alkylamino optionally substituted by 1 to 3 groups R³⁸ or di(C₁₋₆) alkylamino optionally substituted by 1 to 3 groups R³⁸, R¹⁴ is hydrogen, C₁₋₆ alkyl optionally substituted by 1 to 3 groups R³⁸, C₁₋₂₀ alkoxy optionally substituted by 1 to 3 groups R³⁸, C₃₋₆ cycloalkoxy optionally substituted by 1 to 3 groups R³⁸, amino, C₁₋₆ alkylamino optionally substituted by 1 to 3 groups R³⁸ or di(C₁₋₆)alkylamino optionally substituted by 1 to 3 groups R³⁸ and R¹⁵ is hydrogen, C₁₋₂₀ alkoxy optionally substituted by 1 to 3 groups R³⁸, C₃₋₆ cycloalkoxy optionally substituted by 1 to 3 groups R³⁸, C₁₋₁₀ alkylthio optionally substituted by 1 to 3 groups R³⁸, amino, C₁₋₆ alkylamino optionally substituted by 1 to 3 groups R³⁸ or di(C₁₋₆) alkyamino optionally substituted by 1 to 3 groups R³⁸ and each R³⁸ is independently C₁₋₆ alkoxy, phenyl optionally substituted by 1 to 3 groups R³⁹ or heteroaryl optionally substituted by 1 to 3 groups R³⁹, each R³⁹ is independently halogen, cyano, C₁₋₄ alkyl, C₁₋₄ haloalkyl, C₁₋₃ alkoxy(C₁₋₃)alkyl, C₁₋₄ alkoxy, C₁₋₄ haloalkoxy, C₁₋₄ alkylsulphonyl, or C₁₋₄ alkoxycarbonyl and, preferably, each R³⁹ is independently halogen, C₁₋₄ alkyl, C₁₋₄ haloalkyl, C₁₋₄ alkoxy or C₁₋₄ alkoxycarbonyl.

In a yet more preferred embodiment, Z is —C(O)R¹², wherein R¹² is as defined in the paragraph above. In a yet more preferred embodiment, Z is —C(O)R¹², wherein R¹² is hydroxyl, C₁₋₁₀ alkylthio, C₁₋₂₀ alkoxy optionally substituted by 1 or 2 groups R³⁸, C₁₋₁₀ alkenyloxy optionally substituted by 1 or 2 groups R³⁸ or C₁₋₂₀ haloalkoxy optionally substituted by 1 to 2 groups R³⁸ and each R³⁸ is independently as defined above. In a yet more preferred embodiment, Z is —C(O)R¹² wherein R¹² is hydroxyl, C₁₋₁₀ alkoxy, C₁₋₅ alkenyloxy, C₁₋₅ alkoxy(C₁₋₅)alkoxy, or phenyl(C₁₋₂)alkoxy and, most preferably, Z is —C(O)R¹² wherein R¹² is hydroxyl or C₁₋₁₀ alkoxy.

The compounds described below are illustrative of novel compounds of the invention. Table 1 below provides 192 compounds designated compounds 1-1 to 1-192 respectively, of formula (I) wherein X is —NH₂ and Z is —CO₂H.

TABLE 1

| | |
|---|---|
| Compound | Substituent Values |

| Number | A | W | Y |
|---|---|---|---|
| 1-1 | Cl | H | Me |
| 1-2 | Cl | H | CF3 |
| 1-3 | Cl | H | cyclopropyl |
| 1-4 | Cl | H | ethenyl |
| 1-5 | Cl | H | prop-1-enyl |
| 1-6 | Cl | H | 1-methylethenyl |
| 1-7 | Cl | H | prop-2-enyl |
| 1-8 | Cl | H | 2-methylprop-2-enyl |
| 1-9 | Cl | F | Me |
| 1-10 | Cl | F | CF3 |
| 1-11 | Cl | F | cyclopropyl |
| 1-12 | Cl | F | ethenyl |
| 1-13 | Cl | F | prop-1-enyl |
| 1-14 | Cl | F | 1-methylethenyl |
| 1-15 | Cl | F | prop-2-enyl |
| 1-16 | Cl | F | 2-methylprop-2-enyl |
| 1-17 | Cl | Cl | Me |
| 1-18 | Cl | Cl | CF3 |
| 1-19 | Cl | Cl | cyclopropyl |
| 1-20 | Cl | Cl | ethenyl |
| 1-21 | Cl | Cl | prop-1-enyl |
| 1-22 | Cl | Cl | 1-methylethenyl |
| 1-23 | Cl | Cl | prop-2-enyl |
| 1-24 | Cl | Cl | 2-methylprop-2-enyl |
| 1-25 | Cl | Me | Me |
| 1-26 | Cl | Me | CF3 |
| 1-27 | Cl | Me | cyclopropyl |
| 1-28 | Cl | Me | ethenyl |
| 1-29 | Cl | Me | prop-1-enyl |
| 1-30 | Cl | Me | 1-methylethenyl |
| 1-31 | Cl | Me | prop-2-enyl |
| 1-32 | Cl | Me | 2-methylprop-2-enyl |
| 1-33 | F | H | Me |
| 1-34 | F | H | CF3 |
| 1-35 | F | H | cyclopropyl |
| 1-36 | F | H | ethenyl |
| 1-37 | F | H | prop-1-enyl |
| 1-38 | F | H | 1-methylethenyl |
| 1-39 | F | H | prop-2-enyl |
| 1-40 | F | H | 2-methylprop-2-enyl |
| 1-41 | F | F | Me |
| 1-42 | F | F | CF3 |
| 1-43 | F | F | cyclopropyl |
| 1-44 | F | F | ethenyl |
| 1-45 | F | F | prop-1-enyl |
| 1-46 | F | F | 1-methylethenyl |
| 1-47 | F | F | prop-2-enyl |
| 1-48 | F | F | 2-methylprop-2-enyl |
| 1-49 | F | Cl | Me |
| 1-50 | F | Cl | CF3 |
| 1-51 | F | Cl | cyclopropyl |
| 1-52 | F | Cl | ethenyl |
| 1-53 | F | Cl | prop-1-enyl |
| 1-54 | F | Cl | 1-methylethenyl |
| 1-55 | F | Cl | prop-2-enyl |
| 1-56 | F | Cl | 2-methylprop-2-enyl |
| 1-57 | F | Me | Me |
| 1-58 | F | Me | CF3 |
| 1-59 | F | Me | cyclopropyl |
| 1-60 | F | Me | ethenyl |
| 1-61 | F | Me | prop-1-enyl |
| 1-62 | F | Me | 1-methylethenyl |
| 1-63 | F | Me | prop-2-enyl |
| 1-64 | F | Me | 2-methylprop-2-enyl |
| 1-65 | Br | H | Me |
| 1-66 | Br | H | CF3 |
| 1-67 | Br | H | cyclopropyl |
| 1-68 | Br | H | ethenyl |
| 1-69 | Br | H | prop-1-enyl |
| 1-70 | Br | H | 1-methylethenyl |
| 1-71 | Br | H | prop-2-enyl |
| 1-72 | Br | H | 2-methylprop-2-enyl |
| 1-73 | Br | F | Me |
| 1-74 | Br | F | CF3 |
| 1-75 | Br | F | cyclopropyl |
| 1-76 | Br | F | ethenyl |
| 1-77 | Br | F | prop-1-enyl |
| 1-78 | Br | F | 1-methylethenyl |
| 1-79 | Br | F | prop-2-enyl |
| 1-80 | Br | F | 2-methylprop-2-enyl |
| 1-81 | Br | Cl | Me |
| 1-82 | Br | Cl | CF3 |
| 1-83 | Br | Cl | cyclopropyl |
| 1-84 | Br | Cl | ethenyl |
| 1-85 | Br | Cl | prop-1-enyl |
| 1-86 | Br | Cl | 1-methylethenyl |
| 1-87 | Br | Cl | prop-2-enyl |
| 1-88 | Br | Cl | 2-methylprop-2-enyl |
| 1-89 | Br | Me | Me |
| 1-90 | Br | Me | CF3 |
| 1-91 | Br | Me | cyclopropyl |
| 1-92 | Br | Me | ethenyl |
| 1-93 | Br | Me | prop-1-enyl |
| 1-94 | Br | Me | 1-methylethenyl |
| 1-95 | Br | Me | prop-2-enyl |
| 1-96 | Br | Me | 2-methylprop-2-enyl |
| 1-97 | MeS | H | Me |
| 1-98 | MeS | H | CF3 |
| 1-99 | MeS | H | cyclopropyl |
| 1-100 | MeS | H | ethenyl |
| 1-101 | MeS | H | prop-1-enyl |
| 1-102 | MeS | H | 1-methylethenyl |
| 1-103 | MeS | H | prop-2-enyl |
| 1-104 | MeS | H | 2-methylprop-2-enyl |
| 1-105 | MeS | F | Me |
| 1-106 | MeS | F | CF3 |
| 1-107 | MeS | F | cyclopropyl |
| 1-108 | MeS | F | ethenyl |
| 1-109 | MeS | F | prop-1-enyl |
| 1-110 | MeS | F | 1-methylethenyl |
| 1-111 | MeS | F | prop-2-enyl |
| 1-112 | MeS | F | 2-methylprop-2-enyl |
| 1-113 | MeS | Cl | Me |
| 1-114 | MeS | Cl | CF3 |
| 1-115 | MeS | Cl | cyclopropyl |
| 1-116 | MeS | Cl | ethenyl |
| 1-117 | MeS | Cl | prop-1-enyl |
| 1-118 | MeS | Cl | 1-methylethenyl |
| 1-119 | MeS | Cl | prop-2-enyl |
| 1-120 | MeS | Cl | 2-methylprop-2-enyl |
| 1-121 | MeS | Me | Me |
| 1-122 | MeS | Me | CF3 |
| 1-123 | MeS | Me | cyclopropyl |
| 1-124 | MeS | Me | ethenyl |
| 1-125 | MeS | Me | prop-1-enyl |
| 1-126 | MeS | Me | 1-methylethenyl |
| 1-127 | MeS | Me | prop-2-enyl |
| 1-128 | MeS | Me | 2-methylprop-2-enyl |
| 1-129 | 3-chlorophenoxy | H | Me |
| 1-130 | 3-chlorophenoxy | H | CF3 |
| 1-131 | 3-chlorophenoxy | H | cyclopropyl |
| 1-132 | 3-chlorophenoxy | H | ethenyl |
| 1-133 | 3-chlorophenoxy | H | prop-1-enyl |
| 1-134 | 3-chlorophenoxy | H | 1-methylethenyl |
| 1-135 | 3-chlorophenoxy | H | prop-2-enyl |
| 1-136 | 3-chlorophenoxy | H | 2-methylprop-2-enyl |
| 1-137 | 3-chlorophenoxy | F | Me |
| 1-138 | 3-chlorophenoxy | F | CF3 |
| 1-139 | 3-chlorophenoxy | F | cyclopropyl |
| 1-140 | 3-chlorophenoxy | F | ethenyl |
| 1-141 | 3-chlorophenoxy | F | prop-1-enyl |
| 1-142 | 3-chlorophenoxy | F | 1-methylethenyl |
| 1-143 | 3-chlorophenoxy | F | prop-2-enyl |
| 1-144 | 3-chlorophenoxy | F | 2-methylprop-2-enyl |
| 1-145 | 3-chlorophenoxy | Cl | Me |
| 1-146 | 3-chlorophenoxy | Cl | CF3 |
| 1-147 | 3-chlorophenoxy | Cl | cyclopropyl |
| 1-148 | 3-chlorophenoxy | Cl | ethenyl |
| 1-149 | 3-chlorophenoxy | Cl | prop-1-enyl |
| 1-150 | 3-chlorophenoxy | Cl | 1-methylethenyl |
| 1-151 | 3-chlorophenoxy | Cl | prop-2-enyl |
| 1-152 | 3-chlorophenoxy | Cl | 2-methylprop-2-enyl |
| 1-153 | 3-chlorophenoxy | Me | Me |
| 1-154 | 3-chlorophenoxy | Me | CF3 |
| 1-155 | 3-chlorophenoxy | Me | cyclopropyl |
| 1-156 | 3-chlorophenoxy | Me | ethenyl |
| 1-157 | 3-chlorophenoxy | Me | prop-1-enyl |
| 1-158 | 3-chlorophenoxy | Me | 1-methylethenyl |
| 1-159 | 3-chlorophenoxy | Me | prop-2-enyl |
| 1-160 | 3-chlorophenoxy | Me | 2-methylprop-2-enyl |
| 1-161 | Cl | H | CHF2 |
| 1-162 | Cl | H | CHO |
| 1-163 | Cl | H | 2-ethoxyethenyl |
| 1-164 | Cl | H | 1,2-difluoroethenyl |
| 1-165 | Cl | H | 2-methylprop-1-enyl |
| 1-166 | Cl | H | ethynyl |
| 1-167 | Cl | H | prop-1-ynyl |
| 1-168 | Cl | H | 4 chlorophenyl |
| 1-169 | Cl | F | CHF2 |
| 1-170 | Cl | F | CHO |
| 1-171 | Cl | F | 2-ethoxyethenyl |
| 1-172 | Cl | F | 1,2-difluoroethenyl |
| 1-173 | Cl | F | 2-methylprop-1-enyl |
| 1-174 | Cl | F | ethynyl |
| 1-175 | Cl | F | prop-1-ynyl |
| 1-176 | Cl | F | 4 chlorophenyl |
| 1-177 | Cl | Cl | CHF2 |
| 1-178 | Cl | Cl | CHO |
| 1-179 | Cl | Cl | 2-ethoxyethenyl |
| 1-180 | Cl | Cl | 1,2-difluoroethenyl |
| 1-181 | Cl | Cl | 2-methylprop-1-enyl |
| 1-182 | Cl | Cl | ethynyl |
| 1-183 | Cl | Cl | prop-1-ynyl |
| 1-184 | Cl | Cl | 4 chlorophenyl |
| 1-185 | Cl | Me | CHF2 |
| 1-186 | Cl | Me | CHO |
| 1-187 | Cl | Me | 2-ethoxyethenyl |
| 1-188 | Cl | Me | 1,2-difluoroethenyl |
| 1-189 | Cl | Me | 2-methylprop-1-enyl |
| 1-190 | Cl | Me | ethynyl |
| 1-191 | Cl | Me | prop-1-ynyl |
| 1-192 | Cl | Me | 4 chlorophenyl |

192 compounds are described, designated compounds 2-1 to 2-192 respectively, of formula (I) wherein X is NH₂ and Z is CO₂Me, and the values of A, W and Y are as defined in Table 1.

192 compounds are described, designated compounds 3-1 to 3-192 respectively, of formula (I) wherein X is NH₂ and Z is CO₂Et, and the values of A, W and Y are as defined in Table 1.

192 compounds are described, designated compounds 4-1 to 4-192 respectively, of formula (I) wherein X is NH₂ and Z is CO₂CH₂CH₂OEt, and the values of A, W and Y are as defined in Table 1.

192 compounds are described, designated compounds 5-1 to 5-192 respectively, of formula (I) wherein X is NH₂ and Z is CO₂CH₂Ph, and the values of A, W and Y are as defined in Table 1.

192 compounds are described, designated compounds 6-1 to 6-192 respectively, of formula (I) wherein X is NHMe and Z is CO₂H, and the values of A, W and Y are as defined in Table 1.

192 compounds are described, designated compounds 7-1 to 7-192 respectively, of formula (I) wherein X is NHMe and Z is CO₂Me, and the values of A, W and Y are as defined in Table 1.

192 compounds are described, designated compounds 8-1 to 8-192 respectively, of formula (I) wherein X is NMe₂ and Z is CO₂H, and the values of A, W and Y are as defined in Table 1.

192 compounds are described, designated compounds 9-1 to 9-192 respectively, of formula (I) wherein X is NMe₂ and Z is CO₂Me, and the values of A, W and Y are as defined in Table 1.

192 compounds are described, designated compounds 10-1 to 10-192 respectively, of formula (I) wherein X is NHⁱPr and Z is CO₂H, and the values of A, W and Y are as defined in Table 1.

192 compounds are described, designated compounds 11-1 to 11-192 respectively, of formula (I) wherein X is NHⁱPr and Z is CO₂Me, and the values of A, W and Y are as defined in Table 1.

192 compounds are described, designated compounds 12-1 to 12-192 respectively, of formula (I) wherein X is NHprop-2-enyl and Z is CO₂H, and the values of A, W and Y are as defined in Table 1.

192 compounds are described, designated compounds 13-1 to 13-192 respectively, of formula (I) wherein X is NHprop-2-enyl and Z is CO₂Me, and the values of A, W and Y are as defined in Table 1.

192 compounds are described, designated compounds 14-1 to 14-192 respectively, of formula (I) wherein X is NHCOMe and Z is CO₂H, and the values of A, W and Y are as defined in Table 1.

192 compounds are described, designated compounds 15-1 to 15-192 respectively, of formula (I) wherein X is NHCOMe and Z is CO₂Me, and the values of A, W and Y are as defined in Table 1.

192 compounds are described, designated compounds 16-1 to 16-192 respectively, of formula (I) wherein X is NHCO₂Me and Z is CO₂H, and the values of A, W and Y are as defined in Table 1.

192 compounds are described, designated compounds 17-1 to 17-192 respectively, of formula (I) wherein X is NHCO₂Me and Z is CO₂Me, and the values of A, W and Y are as defined in Table 1.

192 compounds are described, designated compounds 18-1 to 18-192 respectively, of formula (I) wherein X is NHSO₂Me and Z is CO₂H, and the values of A, W and Y are as defined in Table 1.

192 compounds are described, designated compounds 19-1 to 19-192 respectively, of formula (I) wherein X is NHSO₂Me and Z is CO₂Me, and the values of A, W and Y are as defined in Table 1.

192 compounds are described, designated compounds 20-1 to 20-192 respectively, of formula (I) wherein X is NHCH₂CH(OH)Me and Z is CO₂H, and the values of A, W and Y are as defined in Table 1.

192 compounds are described, designated compounds 21-1 to 21-192 respectively, of formula (I) wherein X is NHCH₂CH(OH)Me and Z is CO₂Me, and the values of A, W and Y are as defined in Table 1.

192 compounds are described, designated compounds 22-1 to 22-192 respectively, of formula (I) wherein X is NHcyclopropyl and Z is CO₂H, and the values of A, W and Y are as defined in Table 1.

192 compounds are described, designated compounds 23-1 to 23-192 respectively, of formula (I) wherein X is NHcyclopropyl and Z is CO₂Me, and the values of A, W and Y are as defined in Table 1.

192 compounds are described, designated compounds 24-1 to 24-192 respectively, of formula (I) wherein X is NHcyclobutyl and Z is CO₂H, and the values of A, W and Y are as defined in Table 1.

192 compounds are described, designated compounds 25-1 to 25-192 respectively, of formula (I) wherein X is NHcyclobutyl and Z is CO₂Me, and the values of A, W and Y are as defined in Table 1.

192 compounds are described, designated compounds 26-1 to 26-192 respectively, of formula (I) wherein X is NHcyclopentyl and Z is CO₂H, and the values of A, W and Y are as defined in Table 1.

192 compounds are described, designated compounds 27-1 to 27-192 respectively, of formula (I) wherein X is NHcyclopentyl and Z is CO₂Me, and the values of A, W and Y are as defined in Table 1.

192 compounds are described, designated compounds 28-1 to 28-192 respectively, of formula (I) wherein X is NHcyclohexyl and Z is CO₂H, and the values of A, W and Y are as defined in Table 1.

192 compounds are described, designated compounds 29-1 to 29-192 respectively, of formula (I) wherein X is NHcyclohexyl and Z is CO₂Me, and the values of A, W and Y are as defined in Table 1.

192 compounds are described, designated compounds 30-1 to 30-192 respectively, of formula (I) wherein X is 1-pyrrolidinyl and Z is CO₂H, and the values of A, W and Y are as defined in Table 1.

192 compounds are described, designated compounds 31-1 to 31-192 respectively, of formula (I) wherein X is 1-pyrrolidinyl and Z is CO₂Me, and the values of A, W and Y are as defined in Table 1.

192 compounds are described, designated compounds 32-1 to 32-192 respectively, of formula (I) wherein X is NH₂ and Z is CO₂CH₂CH₂OⁿBu, and the values of A, W and Y are as defined in Table 1.

192 compounds are described, designated compounds 33-1 to 33-192 respectively, of formula (I) wherein X is NH₂ and Z is CO₂CH₂CH═CH₂, and the values of A, W and Y are as defined in Table 1.

In a particular embodiment, there is provided a salt of the compound of formula (I) as described or listed above. In particular, the salt of the compound of formula (I) may be derived from an alkali metal, an alkaline earth metal, ammonia or an amine. Preferably, the salt is a sodium salt, a potassium salt or a triethylammonium salt.

General methods for the production of compounds of formula (I) are described below. Unless otherwise stated in the text, the substituents R⁵, R⁶, A, W, X, Y and Z are as defined hereinbefore. The abbreviation LG as used herein refers to any suitable leaving group, and includes halogen, sulphonate, and sulphone groups. The starting materials used for the preparation of the compounds of the invention may be purchased from usual commercial suppliers or may be prepared by known methods. The starting materials as well as the intermediates may be purified before use in the next step by state of the art methodologies such as chromatography, crystallization, distillation and filtration.

Compounds of formula (I) may be prepared from compounds of formula (A) as shown in reaction scheme 1.

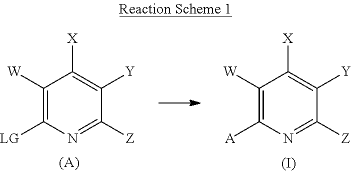

For example (see reaction scheme 2) a compound of formula (I), in which A is a group attached through a sulfur or oxygen atom, may be prepared by reacting an alcohol or thiol (e.g. phenol or thiophenol) with a compound of formula (A) in the presence of a suitable base (e.g. an inorganic base, such as sodium hydride or potassium carbonate) in a suitable solvent (e.g. dimethylformamide). This transformation may also be performed in the presence of a suitable metal (e.g. palladium) catalyst, optionally complexed by any suitable ligands (e.g. phosphine ligands, such as tetrakis(triphenylphosphine)palladium).

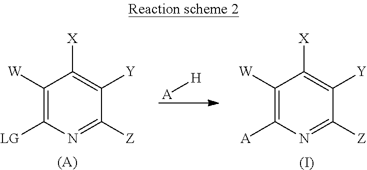

In a second example (see reaction scheme 3) a compound of formula (I), in which A is a group attached through a sulfur or oxygen atom, may be prepared by reacting a metal salt of an alcohol or thiol (e.g. sodium phenoxide or sodium thiophenolate) with a compound of formula (A) in a suitable solvent (e.g. dimethylformamide). This transformation may also be performed in the presence of a suitable metal (e.g. palladium) catalyst, optionally complexed by any suitable ligands (e.g. phosphine ligands, such as tetrakis(triphenylphosphine)palladium).

In a third example (see reaction scheme 3) a compound of formula (I), in which A is a halogen, may be prepared by reacting a metal halide or metalloid derivative A-M (e.g. potassium fluoride, sodium iodide or bromotrimethylsilane) with a compound of formula (A) in a suitable solvent (e.g. acetonitrile or dimethyl sulfoxide).

In a fourth example (see reaction scheme 3) a compound of formula (I), in which A is a cyano group, may be prepared by reacting a metal cyanide (e.g. copper(I) cyanide) with a compound of formula (A) in a suitable solvent (e.g. dimethylformamide or N-methylpyrrolidone). This transformation may also be performed in the presence of a suitable metal (e.g. palladium) catalyst, optionally complexed by any suitable ligands (e.g. phosphine ligands, such as 1,1′-bis(diphenylphosphino)ferrocene).

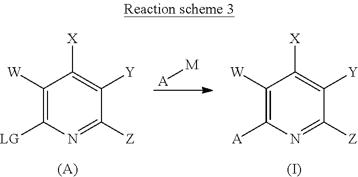

Compounds of formula (I) may be prepared from compounds of formula (B) as shown in reaction scheme 4.

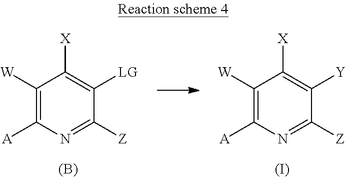

For example (see reaction scheme 5) a compound of formula (I), may be prepared by reacting a suitable metal or metalloid derivative Y-M (e.g. a boronic acid or ester, a trialkyltin derivative, a zinc derivative, a copper derivative or a Grignard reagent) with a compound of formula (B) in the presence of a suitable base (e.g. an inorganic base, such as potassium phosphate or caesium fluoride), a metal source (e.g. a palladium source, such as Pd(OAc)₂) and, optionally, a ligand for the metal (e.g. a phosphine ligand, such as triphenylphosphine) in a suitable solvent (e.g. a single solvent, such as dimethylformamide, or a mixed solvent system, such as a mixture of dimethoxyethane and water or toluene and water). The metal catalyst and ligands may also be added as a single, pre-formed, complex (e.g. a palladium/phosphine complex, such as bis(triphenylphosphine)palladium dichloride or [1,1′-bis(diphenylphosphino)ferrocene]palladium dichloride dichloromethane adduct).

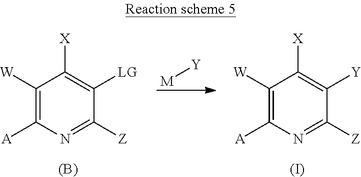

Alternatively a compound of formula (I) in which Y is an alkene or alkyne may be prepared by reacting a compound of formula (B) with an alkene or alkyne in the presence of a suitable base (e.g. an inorganic base, such as potassium phosphate or caesium fluoride or an organic base, such as triethylamine or diisopropylamine), a metal source (e.g. a palladium source, such as Pd(OAc)₂), optionally a second metal source (for example a copper salt, such as copper(I) iodide and, optionally, a ligand for the metal (e.g. a phosphine ligand, such as triphenylphosphine or tris(2-methylphenyl)phosphine) in a suitable solvent (e.g. a single solvent, such as dimethylformamide or acetonitrile, or a mixed solvent system, such as a mixture of dimethoxyethane and water or toluene and water). The metal catalyst and ligands may also be added as a single, pre-formed, complex (e.g. a palladium/phosphine complex, such as tetrakis(triphenylphosphine)palladium or bis(triphenylphosphine)palladium dichloride).

Compounds of formula (I) may be prepared from compounds of formula (C) as shown in reaction scheme 6.

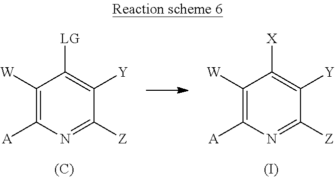

For example (see reaction scheme 7) a compound of formula (I) in which X is NR⁵R⁶, may be prepared from a compound of formula (C) by a reaction with a reagent R⁵R⁶N—H or its salt (e.g. a hydrogen halide salt, such as R⁵R⁶NH₂Cl) in the presence of a suitable base (e.g. an organic base, such as N,N-diisopropylethylamine or an inorganic base, such as potassium carbonate), in a suitable solvent (e.g. an organic solvent, such as dimethylformamide or N-methylpyrrolidone). This transformation may also be performed in the presence of a suitable metal catalyst (e.g. a metal catalyst, such as a palladium source), optionally complexed by any suitable ligands (e.g. phosphine ligand).

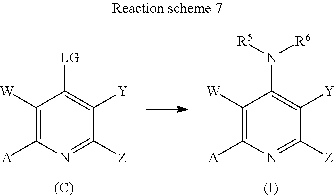

In a second example (see reaction scheme 8) a compound of formula (I) in which X is azide, may be prepared by reacting a metal azide (e.g. sodium azide) with a compound of formula (C) in a suitable solvent (e.g. dimethylformamide).

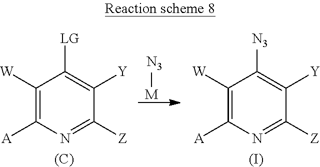

In a third example (see reaction scheme 9) a compound of formula (I), in which X is a group attached through a sulfur or oxygen atom, may be prepared by reacting an alcohol or thiol (e.g. methanol or methanethiol) with a compound of formula (C) in the presence of a suitable base (e.g. an organic base, such as N,N-diisopropylethylamine or an inorganic base, such as potassium carbonate or sodium hydride) in a suitable solvent (e.g. dimethylformamide).

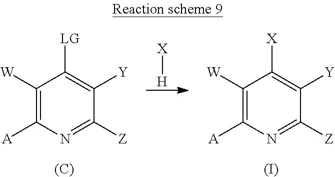

In a fourth example (see reaction scheme 10) a compound of formula (I), in which X is a group attached through a sulfur or oxygen atom, may be prepared by reacting a metal salt of an alcohol or thiol (e.g. sodium methoxide or sodium methanethiolate) with a compound of formula (C) in a suitable solvent (e.g. dimethylformamide).

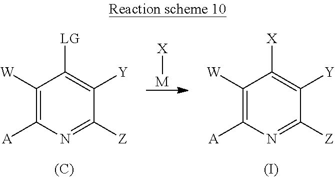

A compound of formula (I) in which X is an amino group, may be prepared from a compound of formula (I) in which X is an azido group, by a reaction with a suitable reducing agent (e.g. sodium borohydride) in a suitable solvent (e.g. methanol) as shown in reaction scheme 11.

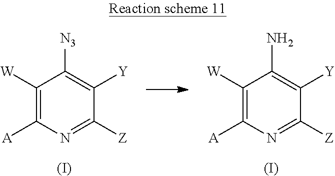

Compounds of formula (I) may be prepared from compounds of formula (D) as shown in reaction scheme 12.

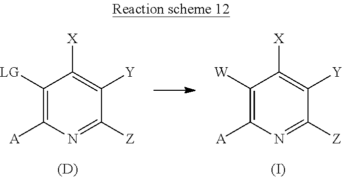

For example (see reaction scheme 13) a compound of formula (I), in which W is a group attached through a carbon atom, may be prepared by reacting a suitable metal or metalloid derivative W-M (e.g. a boronic acid or ester, a trialkyltin derivative, a zinc derivative, a copper derivative or a Grignard reagent) with a compound of formula (D) in the presence of a suitable base (e.g. an inorganic base, such as potassium phosphate or caesium fluoride), a metal source (e.g. a palladium source, such as Pd(OAc)₂) and, optionally, a ligand for the metal (e.g. a phosphine ligand, such as triphenylphosphine) in a suitable solvent (e.g. a single solvent, such as dimethylformamide, or a mixed solvent system, such as a mixture of dimethoxyethane and water or toluene and water). The metal catalyst and ligands may also be added as a single, pre-formed, complex (e.g. a palladium/phosphine complex, such as bis(triphenylphosphine)palladium dichloride or [1,1′-bis(diphenylphosphino)ferrocene]palladium dichloride dichloromethane adduct).

In a second example (see reaction scheme 13) a compound of formula (I), in which W is a cyano group, may be prepared by reacting a metal cyanide (e.g. copper(I) cyanide) with a compound of formula (D) in a suitable solvent (e.g. dimethylformamide or N-methylpyrrolidone). This transformation may also be performed in the presence of a suitable metal (e.g. palladium) catalyst, optionally complexed by any suitable ligands (e.g. phosphine ligands, such as 1,1′-bis(diphenylphosphino)ferrocene).

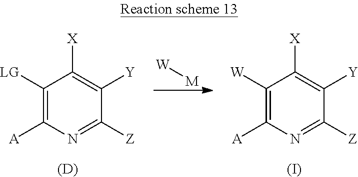

Compounds of formula (D) may be prepared from compounds of formula (E) as shown in reaction scheme 14.

For example (see reaction scheme 14) a compound of formula (D) in which LG is a halogen may be prepared from a compound of formula (E) by reaction with a halogenating agent (e.g. Selectfluor®, an N-halosuccinimide such as N-chlorosuccinimide or N-iodosuccinimide, or an elemental halogen such as bromine) in a suitable solvent (e.g. acetonitrile). This transformation may also be performed by first deprotonating the compound of formula (E) with a suitable base, followed by a reaction with a halogenating agent (e.g. Selectfluor®, an N-halosuccinimide such as N-chlorosuccinimide or N-iodosuccinimide, or an elemental halogen such as bromine).

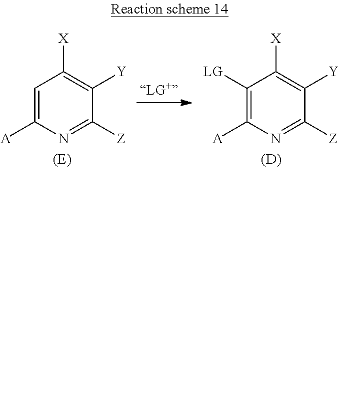

Compounds of formulae (A), (B), (C) and (E) may be prepared from compounds of formula (G) by suitable combinations of the methods described above. Compounds of formula (D) may be prepared from compounds of formula (H) by suitable combinations of the methods described above (see reaction scheme 15).

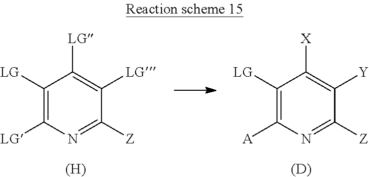

Compounds of formula (H) are known in the literature or can be made from compounds know in the literature by standard methods.

Compounds of formula (G) may be prepared from compounds of formula (F) as shown in reaction scheme 16. A compound of formula (G) in which LG is a halogen may be prepared from a compound of formula (F) by treatment with a suitable reagent (e.g. a phosphoryl halide such as phosphorous oxychloride).

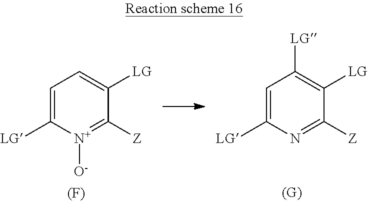

Compounds of formula (F) may be prepared from compounds of formula (J) as shown in reaction scheme 17. A compound of formula (F) in which LG is a halogen may be prepared from a compound of formula (J) by treatment with a suitable oxidizing agent (e.g. urea hydrogen peroxide) and a suitable acid anhydride (e.g. trifluoroacetic anhydride) in a suitable solvent (e.g. dichloromethane).

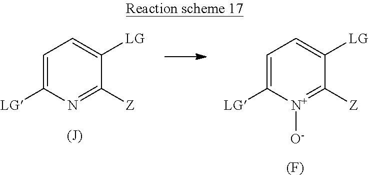

Compounds of formula (J) may be prepared from compounds of formula (K) as shown in reaction scheme 18. A compound of formula (J) in which LG is a halogen may be prepared from a compound of formula (K) by treatment with a suitable reagent (e.g. a phosphoryl halide such as phosphorous oxychloride).

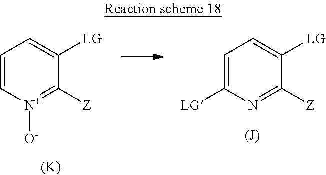

Compounds of formula (K) may be prepared from compounds of formula (L) as shown in reaction scheme 19. A compound of formula (K) in which LG is a halogen may be prepared from a compound of formula (L) by treatment with a suitable oxidizing agent (e.g. urea hydrogen peroxide) and a suitable acid anhydride (e.g. trifluoroacetic anhydride) in a suitable solvent (e.g. dichloromethane).

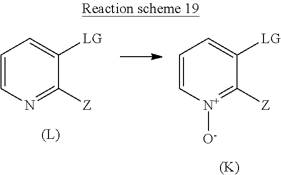

Compounds of formula (L) may be prepared from compounds of formula (M) as shown in reaction scheme 20. A compound of formula (L) in which Z is an ester may be prepared from a compound of formula (M) by treatment with a catalytic amount of suitable acid (e.g. concentrated sulphuric acid) in a suitable solvent (e.g. methanol).

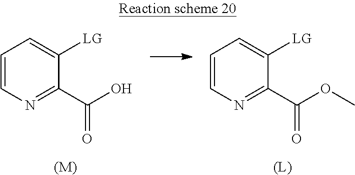

Compounds of formula (M) may be prepared from compounds of formula (N) as shown in reaction scheme 21. A compound of formula (M) may be prepared from a compound of formula (N) by treatment with a suitable base (e.g. lithium tetramethylpiperidide) in a suitable solvent (e.g. tetrahydrofuran) followed by a treatment with a halogenating agent (e.g. iodine or hexachloroethane).

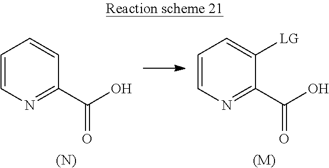

One skilled in the art will realise that it is often possible to alter the order in which the transformations described above are conducted, or to combine them in alternative ways to prepare a wide range of compounds of formula (I). All such variations are contemplated within the scope of the invention.

The skilled man will also be aware that some reagents will be incompatible with certain values or combinations of the substituents R⁵, R⁶, A, W, X, Y and Z as defined herein, and any additional steps, such as protection and/or deprotection steps, which are necessary to achieve the desired transformation will be clear to the skilled man.

Compounds of formula (I) may be used in unmodified form, i.e. as obtainable from synthesis, but preferably are formulated in any suitable manner using formulation adjuvants, such as carriers, solvents and surface-active substances, for example, as described hereinafter. Accordingly, the present invention provides a herbicidal formulation comprising a compound of formula (I) together with at least one agriculturally acceptable adjuvant or diluent.

The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, suspension concentrates, emulsifiable granules, soluble liquids, water-soluble concentrates (with water or a water-miscible organic solvent as carrier), impregnated polymer films or in other forms known e.g. from the Manual on Development and Use of FAO Specifications for Plant Protection Products, 5th Edition, 1999. The formulations can be in the form of concentrates which are diluted prior to use, although ready-to-use formulations can also be made. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof. The active ingredients can also be contained in very fine microcapsules consisting of a polymer. Microcapsules usually have a diameter of from 0.1 to 500 microns. Typically, they will contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other known polymers. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid (e.g. butyl acetate, ethyl acetate, isoamyl acetate, amyl acetate), diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol (PEG), propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, methanol, ethanol, isopropanol, and alcohols of higher molecular weight, such as amyl alcohol, tetrahydrofurfuryl alcohol, hexanol, octanol, N-methyl-2-pyrrolidone and the like. Water is generally the carrier of choice for diluting the concentrates. Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances, as described, for example, in CFR 180.1001. (c) & (d).

A large number of surface-active substances may advantageously be used in the formulations, especially in those formulations designed to be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in “McCutcheon's Detergents and Emulsifiers Annual” MC Publishing Corp., Ridgewood N.J., 1981.

Further adjuvants that can usually be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and also liquid and solid fertilisers.

The compositions according to the invention can additionally include an additive comprising an oil of vegetable or animal origin, a mineral oil, alkyl esters of such oils or mixtures of such oils and oil derivatives. The amount of oil additive in the composition according to the invention is generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil, olive oil or sunflower oil, emulsified vegetable oil, such as AMIGO® (Rhone-Poulenc Canada Inc.), alkyl esters of oils of vegetable origin, for example the methyl derivatives, or an oil of animal origin, such as fish oil or beef tallow. A preferred additive contains, for example, as active components essentially 80% by weight alkyl esters of fish oils and 15% by weight methylated rapeseed oil, and also 5% by weight of customary emulsifiers and pH modifiers. Especially preferred oil additives comprise alkyl esters of C₈₋₂₂ fatty acids, especially the methyl derivatives of C₁₂₋₁₈ fatty acids, for example the methyl esters of lauric acid, palmitic acid and oleic acid, being of importance. Those esters are known as methyl laurate (CAS-111-82-0), methyl palmitate (CAS-112-39-0) and methyl oleate (CAS-112-62-9). A preferred fatty acid methyl ester derivative is Emery® 2230 and 2231 (Cognis GmbH). Those and other oil derivatives are also known from the Compendium of Herbicide Adjuvants, 5th Edition, Southern Illinois University, 2000. Another preferred adjuvant is Adigor® (Syngenta AG) which is a methylated rapeseed oil-based adjuvant.

The application and action of the oil additives can be further improved by combination with surface-active substances, such as non-ionic, anionic or cationic surfactants. Examples of suitable anionic, non-ionic and cationic surfactants are listed on pages 7 and 8 of WO97/34485. Preferred surface-active substances are anionic surfactants of the dodecylbenzylsulfonate type, especially the calcium salts thereof, and also non-ionic surfactants of the fatty alcohol ethoxylate type. Special preference is given to ethoxylated C₁₂₋₂₂ fatty alcohols having a degree of ethoxylation of from 5 to 40. Examples of commercially available surfactants are the Genapol types (Clariant AG). Also preferred are silicone surfactants, especially polyalkyl-oxide-modified heptamethyltriloxanes which are commercially available e.g. as Silwet L-77®, and also perfluorinated surfactants. The concentration of the surface-active substances in relation to the total additive is generally from 1 to 30% by weight. Examples of oil additives consisting of mixtures of oil or mineral oils or derivatives thereof with surfactants are Edenor ME SU®, Turbocharge® (Syngenta AG, CH) or ActipronC (BP Oil UK Limited, GB).

If desired, it is also possible for the mentioned surface-active substances to be used in the formulations on their own, that is to say without oil additives.

Furthermore, the addition of an organic solvent to the oil additive/surfactant mixture may contribute to an additional enhancement of action. Suitable solvents are, for example, Solvesso® (ESSO) or Aromatic Solvent® (Exxon Corporation). The concentration of such solvents can be from 10 to 80% by weight of the total weight. Oil additives that are present in admixture with solvents are described, for example, in U.S. Pat. No. 4,834,908. A commercially available oil additive disclosed therein is known by the name MERGE® (BASF Corporation). A further oil additive that is preferred according to the invention is SCORE® (Syngenta Crop Protection Canada).

In addition to the oil additives listed above, for the purpose of enhancing the action of the compositions according to the invention it is also possible for formulations of alkylpyrrolidones (e.g. Agrimax®) to be added to the spray mixture. Formulations of synthetic lattices, e.g. polyacrylamide, polyvinyl compounds or poly-1-p-menthene (e.g. Bond®, Courier® or Emerald®) may also be used. It is also possible for solutions that contain propionic acid, for example Eurogkem Pen-e-trate®, to be added to the spray mixture as action-enhancing agent.

Herbicidal compositions of the invention generally comprise from 0.1 to 99% by weight, especially from 0.1 to 95% by weight, compounds of formula (I) and from 1 to 99.9% by weight of a formulation adjuvant which preferably includes from 0 to 25% by weight of a surface-active substance. Whereas commercial products will preferably be formulated as concentrates, the end user will normally employ dilute formulations.

Examples of preferred formulation types and their typical compositions are given below (% is percent by weight). Wettable powders as described herein are one particularly preferred type of formulation for use in the invention. In other preferred embodiments, in particular where the compound/composition/formulation of the invention is intended for use on turf, granular (inert or fertiliser) formulations as described herein are particularly suitable.

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%

The following Examples further illustrate, but do not limit, the invention.

### Formulation Examples for Herbicides of Formula (I) (%=% by Weight)

F1. Emulsifiable concentrates

| | a) | b) | c) | d) |
|---|---|---|---|---|
| active ingredient | 5% | 10% | 25% | 50% |
| calcium dodecylbenzenesulfonate | 6% | 8% | 6% | 8% |
| castor oil polyglycol ether | 4% | — | 4% | 4% |
| (36 mol of ethylene oxide) | | | | |
| octylphenol polyglycol ether | — | 4% | — | 2% |
| (7-8 mol of ethylene oxide) | | | | |
| N-Methyl pyrrolidone | — | — | 10% | 20% |
| arom. hydrocarbon mixture | 85% | 78% | 55% | 16% |
| (C₉-C₁₂) | | | | |

Emulsions of any desired concentration can be obtained from such concentrates by dilution with water.

F2. Solutions

| | a) | b) | c) | d) |
|---|---|---|---|---|
| active ingredient | 5% | 10% | 50% | 90% |
| 1-methoxy-3-(3-methoxy- | — | 20% | 20% | — |
| propoxy)-propane | | | | |
| polyethylene glycol MW 400 | 20% | 10% | — | — |
| NMP | — | — | 30% | 10% |
| arom. hydrocarbon mixture | 75% | 60% | — | — |
| (C₉-C₁₂) | | | | |

The solutions are suitable for use in the form of microdrops.

F3. Wettable powders

| | a) | b) | c) | d) |
|---|---|---|---|---|
| active ingredient | 5% | 25% | 50% | 80% |
| sodium lignosulfonate | 4% | — | 3% | — |
| sodium lauryl sulfate | 2% | 3% | — | 4% |
| sodium diisobutylnaphthalene- | — | 6% | 5% | 6% |
| sulfonate | | | | |
| octylphenol polyglycol ether | — | 1% | 2% | — |
| (7-8 mol of ethylene oxide) | | | | |
| highly dispersed silicic acid | 1% | 3% | 5% | 10% |
| kaolin | 88% | 62% | 35% | — |

The active ingredient is mixed thoroughly with the adjuvants and the mixture is thoroughly ground in a suitable mill, affording wettable powders which can be diluted with water to give suspensions of any desired concentration.

F4. Coated granules

| | a) | b) | c) |
|---|---|---|---|
| active ingredient | 0.1% | 5% | 15% |
| highly dispersed silicic acid | 0.9% | 2% | 2% |
| inorganic carrier | 99.0% | 93% | 83% |
| (diameter 0.1-1 mm) | | | |
| e.g. CaCO₃ or SiO₂ | | | |

The active ingredient is dissolved in methylene chloride and applied to the carrier by spraying, and the solvent is then evaporated off in vacuo.

F5. Coated granules

| | a) | b) | c) |
|---|---|---|---|
| active ingredient | 0.1% | 5% | 15% |
| polyethylene glycol MW 200 | 1.0% | 2% | 3% |
| highly dispersed silicic acid | 0.9% | 1% | 2% |
| inorganic carrier | 98.0% | 92% | 80% |
| (diameter 0.1-1 mm) | | | |
| e.g. CaCO₃ or SiO₂ | | | |

The finely ground active ingredient is uniformly applied, in a mixer, to the carrier moistened with polyethylene glycol. Non-dusty coated granules are obtained in this manner.

F6. Extruder granules

| | a) | b) | c) | d) |
|---|---|---|---|---|
| active ingredient | 0.1% | 3% | 5% | 15% |
| sodium lignosulfonate | 1.5% | 2% | 3% | 4% |
| carboxymethylcellulose | 1.4% | 2% | 2% | 2% |
| kaolin | 97.0% | 93% | 90% | 79% |

The active ingredient is mixed and ground with the adjuvants, and the mixture is moistened with water. The mixture is extruded and then dried in a stream of air.

F7. Dusts

| | a) | b) | c) |
|---|---|---|---|
| active ingredient | 0.1% | 1% | 5% |
| talcum | 39.9% | 49% | 35% |
| kaolin | 60.0% | 50% | 60% |

Ready-to-use dusts are obtained by mixing the active ingredient with the carriers and grinding the mixture in a suitable mill.

F8. Suspension concentrates

| | a) | b) | c) | d) |
|---|---|---|---|---|
| active ingredient | 3% | 10% | 25% | 50% |
| ethylene glycol | 5% | 5% | 5% | 5% |
| nonylphenol polyglycol ether | — | 1% | 2% | — |
| (15 mol of ethylene oxide) | | | | |
| sodium lignosulfonate | 3% | 3% | 4% | 5% |
| carboxymethylcellulose | 1% | 1% | 1% | 1% |
| 37% aqueous formaldehyde | 0.2% | 0.2% | 0.2% | 0.2% |
| solution | | | | |
| silicone oil emulsion | 0.8% | 0.8% | 0.8% | 0.8% |
| water | 87% | 79% | 62% | 38% |

The finely ground active ingredient is intimately mixed with the adjuvants, giving a suspension concentrate from which suspensions of any desired concentration can be obtained by dilution with water.

Compounds of the invention (as well as mixtures and/or formulations containing the same) find utility as herbicides, and may thus be employed in methods of controlling plant growth. Such methods involve applying to the plants or to the locus thereof a herbicidally effective amount of said compound, or composition comprising the same (or mixture as described hereinafter). The invention thus also relates to a method of inhibiting plant growth which comprises applying to the plants or to the locus thereof a herbicidally effective amount of a compound of formula (I), composition, or mixture of the invention. In particular the invention provides a method of controlling weeds in crops of useful plants, which comprises applying to said weeds or the locus of said weeds, or to said crop of useful plants, a compound of formula I or a composition or mixture containing the same.

The term “locus” as used herein includes not only areas where weeds may already be growing, but also areas where weeds have yet to emerge, and also to areas under cultivation with respect to crops of useful plants. Areas under cultivation include land on which the crop plants are already growing and land intended for cultivation with such crop plants.

A compound, composition, and/or mixture of the invention may be used in a pre-emergence application and/or in a post-emergence application in order to mediate its effect.

Crops of useful plants in which compounds of formula (I), as well as formulations and/or mixtures containing the same, may be used according to the invention include perennial crops, such as citrus fruit, grapevines, nuts, oil palms, olives, pome fruit, stone fruit and rubber, and annual arable crops, such as cereals, for example barley and wheat, cotton, oilseed rape, maize, rice, soy beans, sugar beet, sugar cane, sunflowers, ornamentals and vegetables, especially cereals and maize.

Compounds of formula (I), formulations and/or mixtures containing the same may also be used on turf, pasture, rangeland, rights of way etc. In particular they may be used on golf-courses, lawns, parks, sports-fields, race-courses and the like.

Crops are to be understood as also including those crops which have been rendered tolerant to herbicides or classes of herbicides (e.g. ALS-, GS-, EPSPS-, PPO- and HPPD-inhibitors and synthetic auxins) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood as being those which are obtained by conventional methods of breeding or genetic engineering and contain so-called output traits (e.g. improved storage stability, higher nutritional value and improved flavour).

The term “weeds” as used herein means any undesired plant, and thus includes not only agronomically important weeds as described below, but also volunteer crop plants.

Compounds of formula (I) may be used against a large number of agronomically important weeds. The weeds that may be controlled include both monocotyledonous and dicotyledonous weeds, such as, for example, Alisma spp, Leptochloa chinensis, Stellaria, Nasturtium, Agrostis, Digitaria, Avena, Setaria, Sinapis, Lolium, Solanum, Echinochloa, Scirpus, Monochoria, Sagittaria, Bromus, Alopecurus, Sorghum, Rottboellia, Cyperus and especially Cyperus iria, Abutilon, Sida, Xanthium, Amaranthus, Chenopodium, Ipomoea, Chrysanthemum, Galium, Viola, Veronica, Bidens, Euphorbia, Ischaemum, Polygonum, Helianthus, Panicum, Eriochloa, Brachiaria, Cenchrus, Commelina, Spermacoce, Senna, Tridax, Richardia, Chamaesyce, and Conyza spp.

The rates of application of compounds of formula (I) may vary within wide limits and depend on the nature of the soil, the method of application (pre- or post-emergence; seed dressing; application to the seed furrow; no tillage application etc.), the crop plant, or weed to be controlled, the prevailing climatic conditions, and other factors governed by the method of application, the time of application and the target crop. The compounds of formula I according to the invention are generally applied at a rate of from 10 to 2000 g/ha, especially from 25 to 1000 g/ha.

Any method of application to weeds/crop of useful plant, or locus thereof, which is routinely used in agriculture may be used, for example application by spray or broadcast method typically after suitable dilution of a compound of formula (I) (whether said compound is formulated and/or in combination with one or more further active ingredients and/or safeners, as described herein).

The compounds of formula (I) according to the invention can also be used in combination with other active ingredients, e.g. other herbicides, and/or insecticides, and/or acaricides, and/or nematocides, and/or molluscicides, and/or fungicides, and/or plant growth regulators. Such mixtures, and the use of such mixtures to control weeds and/or undesired plant growth form yet further aspects of the invention. For the avoidance of doubt, mixtures of invention also include mixtures of two or more different compounds of formula (I). In particular, the present invention also relates to a composition of the invention which comprises at least one further herbicide in addition to the compound of formula (I).

Where a compound of formula (I) is combined with at least one additional herbicide, the following mixtures of the compound of formula (I) are particularly preferred. Compound of formula (I)+acetochlor, compound of formula (I)+acifluorfen, compound of formula (I)+acifluorfen-sodium, compound of formula (I)+aclonifen, compound of formula (I)+acrolein, compound of formula (I)+alachlor, compound of formula (I)+alloxydim, compound of formula (I)+allyl alcohol, compound of formula (I)+ametryn, compound of formula (I)+amicarbazone, compound of formula (I)+amidosulfuron, compound of formula (I)+aminocyclopyrachlor, compound of formula (I)+aminopyralid, compound of formula (I)+amitrole, compound of formula (I)+ammonium sulfamate, compound of formula (I)+anilofos, compound of formula (I)+asulam, compound of formula (I)+atrazine, formula (I)+aviglycine, formula (I)+azafenidin, compound of formula (I)+azimsulfuron, compound of formula (I)+BCPC, compound of formula (I)+beflubutamid, compound of formula (I)+benazolin, formula (I)+bencarbazone, compound of formula (I)+benfluralin, compound of formula (I)+benfuresate, compound of formula (I)+bensulfuron, compound of formula (I)+bensulfuron-methyl, compound of formula (I)+bensulide, compound of formula (I)+bentazone, compound of formula (I)+benzfendizone, compound of formula (I)+benzobicyclon, compound of formula (I)+benzofenap, compound of formula (I)+bifenox, compound of formula (I)+bilanafos, compound of formula (I)+bispyribac, compound of formula (I)+bispyribac-sodium, compound of formula (I)+borax, compound of formula (I)+bromacil, compound of formula (I)+bromobutide, formula (I)+bromophenoxim, compound of formula (I)+bromoxynil, compound of formula (I)+butachlor, compound of formula (I)+butafenacil, compound of formula (I)+butamifos, compound of formula (I)+butralin, compound of formula (I)+butroxydim, compound of formula (I)+butylate, compound of formula (I)+cacodylic acid, compound of formula (I)+calcium chlorate, compound of formula (I)+cafenstrole, compound of formula (I)+carbetamide, compound of formula (I)+carfentrazone, compound of formula (I)+carfentrazone-ethyl, compound of formula (I)+CDEA, compound of formula (I)+CEPC, compound of formula (I)+chlorflurenol, compound of formula (I)+chlorflurenol-methyl, compound of formula (I)+chloridazon, compound of formula (I)+chlorimuron, compound of formula (I)+chlorimuron-ethyl, compound of formula (I)+chloroacetic acid, compound of formula (I)+chlorotoluron, compound of formula (I)+chlorpropham, compound of formula (I)+chlorsulfuron, compound of formula (I)+chlorthal, compound of formula (I)+chlorthal-dimethyl, compound of formula (I)+cinidon-ethyl, compound of formula (I)+cinmethylin, compound of formula (I)+cinosulfuron, compound of formula (I)+cisanilide, compound of formula (I)+clethodim, compound of formula (I)+clodinafop, compound of formula (I)+clodinafop-propargyl, compound of formula (I)+clomazone, compound of formula (I)+clomeprop, compound of formula (I)+clopyralid, compound of formula (I)+cloransulam, compound of formula (I)+cloransulam-methyl, compound of formula (I)+CMA, compound of formula (I)+4-CPB, compound of formula (I)+CPMF, compound of formula (I)+4-CPP, compound of formula (I)+CPPC, compound of formula (I)+cresol, compound of formula (I)+cumyluron, compound of formula (I)+cyanamide, compound of formula (I)+cyanazine, compound of formula (I)+cycloate, compound of formula (I)+cyclosulfamuron, compound of formula (I)+cycloxydim, compound of formula (I)+cyhalofop, compound of formula (I)+cyhalofop-butyl, compound of formula (I)+2,4-D, compound of formula (I)+3,4-DA, compound of formula (I)+daimuron, compound of formula (I)+dalapon, compound of formula (I)+dazomet, compound of formula (I)+2,4-DB, compound of formula (I)+3,4-DB, compound of formula (I)+2,4-DEB, compound of formula (I)+desmedipham, formula (I)+desmetryn, compound of formula (I)+dicamba, compound of formula (I)+dichlobenil, compound of formula (I)+ortho-dichlorobenzene, compound of formula (I)+para-dichlorobenzene, compound of formula (I)+dichlorprop, compound of formula (I)+dichlorprop-P, compound of formula (I)+diclofop, compound of formula (I)+diclofop-methyl, compound of formula (I)+diclosulam, compound of formula (I)+difenzoquat, compound of formula (I)+difenzoquat metilsulfate, compound of formula (I)+diflufenican, compound of formula (I)+diflufenzopyr, compound of formula (I)+dimefuron, compound of formula (I)+dimepiperate, compound of formula (I)+dimethachlor, compound of formula (I)+dimethametryn, compound of formula (I)+dimethenamid, compound of formula (I)+dimethenamid-P, compound of formula (I)+dimethipin, compound of formula (I)+dimethylarsinic acid, compound of formula (I)+dinitramine, compound of formula (I)+dinoterb, compound of formula (I)+diphenamid, formula (I)+dipropetryn, compound of formula (I)+diquat, compound of formula (I)+diquat dibromide, compound of formula (I)+dithiopyr, compound of formula (I)+diuron, compound of formula (I)+DNOC, compound of formula (I)+3,4-DP, compound of formula (I)+DSMA, compound of formula (I)+EBEP, compound of formula (I)+endothal, compound of formula (I)+EPTC, compound of formula (I)+esprocarb, compound of formula (I)+ethalfluralin, compound of formula (I)+ethametsulfuron, compound of formula (I)+ethametsulfuron-methyl, formula (I)+ethephon, compound of formula (I)+ethofumesate, compound of formula (I)+ethoxyfen, compound of formula (I)+ethoxysulfuron, compound of formula (I)+etobenzanid, compound of formula (I)+fenoxaprop, compound of formula (I)+fenoxaprop-P, compound of formula (I)+fenoxaprop-ethyl, compound of formula (I)+fenoxaprop-P-ethyl, compound of formula (I)+fentrazamide, compound of formula (I)+ferrous sulfate, compound of formula (I)+flamprop-M, compound of formula (I)+flazasulfuron, compound of formula (I)+florasulam, compound of formula (I)+fluazifop, compound of formula (I)+fluazifop-butyl, compound of formula (I)+fluazifop-P, compound of formula (I)+fluazifop-P-butyl, formula (I)+fluazolate, compound of formula (I)+flucarbazone, compound of formula (I)+flucarbazone-sodium, compound of formula (I)+flucetosulfuron, compound of formula (I)+fluchloralin, compound of formula (I)+flufenacet, compound of formula (I)+flufenpyr, compound of formula (I)+flufenpyr-ethyl, formula (I)+flumetralin, compound of formula (I)+flumetsulam, compound of formula (I)+flumiclorac, compound of formula (I)+flumiclorac-pentyl, compound of formula (I)+flumioxazin, formula (I)+flumipropin, compound of formula (I)+fluometuron, compound of formula (I)+fluoroglycofen, compound of formula (I)+fluoroglycofen-ethyl, formula (I)+fluoxaprop, formula (I)+flupoxam, formula (I)+flupropacil, compound of formula (I)+flupropanate, compound of formula (I)+flupyrsulfuron, compound of formula (I)+flupyrsulfuron-methyl-sodium, compound of formula (I)+flurenol, compound of formula (I)+fluridone, compound of formula (I)+fluorochloridone, compound of formula (I)+fluoroxypyr, compound of formula (I)+flurtamone, compound of formula (I)+fluthiacet, compound of formula (I)+fluthiacet-methyl, compound of formula (I)+fomesafen, compound of formula (I)+foramsulfuron, compound of formula (I)+fosamine, compound of formula (I)+glufosinate, compound of formula (I)+glufosinate-ammonium, compound of formula (I)+glyphosate, compound of formula (I)+halosulfuron, compound of formula (I)+halosulfuron-methyl, compound of formula (I)+haloxyfop, compound of formula (I)+haloxyfop-P, compound of formula (I)+HC-252, compound of formula (I)+hexazinone, compound of formula (I)+imazamethabenz, compound of formula (I)+imazamethabenz-methyl, compound of formula (I)+imazamox, compound of formula (I)+imazapic, compound of formula (I)+imazapyr, compound of formula (I)+imazaquin, compound of formula (I)+imazethapyr, compound of formula (I)+imazosulfuron, compound of formula (I)+indanofan, compound of formula (I)+iodomethane, compound of formula (I)+iodosulfuron, compound of formula (I)+iodosulfuron-methyl-sodium, compound of formula (I)+ioxynil, compound of formula (I) and ipfencarbazone, compound of formula (I)+isoproturon, compound of formula (I)+isouron, compound of formula (I)+isoxaben, compound of formula (I)+isoxachlortole, compound of formula (I)+isoxaflutole, formula (I)+isoxapyrifop, compound of formula (I)+karbutilate, compound of formula (I)+lactofen, compound of formula (I)+lenacil, compound of formula (I)+linuron, compound of formula (I)+MAA, compound of formula (I)+MAMA, compound of formula (I)+MCPA, compound of formula (I)+MCPA-thioethyl, compound of formula (I)+MCPB, compound of formula (I)+mecoprop, compound of formula (I)+mecoprop-P, compound of formula (I)+mefenacet, compound of formula (I)+mefluidide, compound of formula (I)+mesosulfuron, compound of formula (I)+mesosulfuron-methyl, compound of formula (I)+mesotrione, compound of formula (I)+metam, compound of formula (I)+metamifop, compound of formula (I)+metamitron, compound of formula (I)+metazachlor, compound of formula (I) and metazosulfuron, compound of formula (I)+methabenzthiazuron, formula (I)+methazole, a compound of formula (I) and methiozolin, compound of formula (I)+methylarsonic acid, compound of formula (I)+methyldymron, compound of formula (I)+methyl isothiocyanate, compound of formula (I)+metobenzuron, formula (I)+metobromuron, compound of formula (I)+metolachlor, compound of formula (I)+S-metolachlor, compound of formula (I)+metosulam, compound of formula (I)+metoxuron, compound of formula (I)+metribuzin, compound of formula (I)+metsulfuron, compound of formula (I)+metsulfuron-methyl, compound of formula (I)+MK-616, compound of formula (I)+molinate, compound of formula (I)+monolinuron, a compound of formula (I) and monosulfuron, a compound of formula (I) and monosulfuron-ester compound of formula (I)+MSMA, compound of formula (I)+naproanilide, compound of formula (I)+napropamide, compound of formula (I)+naptalam, formula (I)+NDA-402989, compound of formula (I)+neburon, compound of formula (I)+nicosulfuron, formula (I)+nipyraclofen, formula (I)+n-methyl glyphosate, compound of formula (I)+nonanoic acid, compound of formula (I)+norflurazon, compound of formula (I)+oleic acid (fatty acids), compound of formula (I)+orbencarb, compound of formula (I)+orthosulfamuron, compound of formula (I)+oryzalin, compound of formula (I)+oxadiargyl, compound of formula (I)+oxadiazon, compound of formula (I)+oxasulfuron, compound of formula (I)+oxaziclomefone, compound of formula (I)+oxyfluorfen, compound of formula (I)+paraquat, compound of formula (I)+paraquat dichloride, compound of formula (I)+pebulate, compound of formula (I)+pendimethalin, compound of formula (I)+penoxsulam, compound of formula (I)+pentachlorophenol, compound of formula (I)+pentanochlor, compound of formula (I)+pentoxazone, compound of formula (I)+pethoxamid, compound of formula (I)+petrolium oils, compound of formula (I)+phenmedipham, compound of formula (I)+phenmedipham-ethyl, compound of formula (I)+picloram, compound of formula (I)+picolinafen, compound of formula (I)+pinoxaden, compound of formula (I)+piperophos, compound of formula (I)+potassium arsenite, compound of formula (I)+potassium azide, compound of formula (I)+pretilachlor, compound of formula (I)+primisulfuron, compound of formula (I)+primisulfuron-methyl, compound of formula (I)+prodiamine, compound of formula (I)+profluazol, compound of formula (I)+profoxydim, formula (I)+prohexadione-calcium, compound of formula (I)+prometon, compound of formula (I)+prometryn, compound of formula (I)+propachlor, compound of formula (I)+propanil, compound of formula (I)+propaquizafop, compound of formula (I)+propazine, compound of formula (I)+propham, compound of formula (I)+propisochlor, compound of formula (I)+propoxycarbazone, compound of formula (I)+propoxycarbazone-sodium, compound of formula (I)+propyzamide, compound of formula (I)+prosulfocarb, compound of formula (I)+prosulfuron, compound of formula (I)+pyraclonil, compound of formula (I)+pyraflufen, compound of formula (I)+pyraflufen-ethyl, formula (I)+pyrasulfotole, compound of formula (I)+pyrazolynate, compound of formula (I)+pyrazosulfuron, compound of formula (I)+pyrazosulfuron-ethyl, compound of formula (I)+pyrazoxyfen, compound of formula (I)+pyribenzoxim, compound of formula (I)+pyributicarb, compound of formula (I)+pyridafol, compound of formula (I)+pyridate, compound of formula (I)+pyriftalid, compound of formula (I)+pyriminobac, compound of formula (I)+pyriminobac-methyl, compound of formula (I)+pyrimisulfan, compound of formula (I)+pyrithiobac, compound of formula (I)+pyrithiobac-sodium, formula (I)+pyroxasulfone, formula (I)+pyroxulam, compound of formula (I)+quinclorac, compound of formula (I)+quinmerac, compound of formula (I)+quinoclamine, compound of formula (I)+quizalofop, compound of formula (I)+quizalofop-P, compound of formula (I)+quizalofop-ethyl, compound of formula (I)+quizalofop-P-ethyl, compound of formula (I)+rimsulfuron, compound of formula (I)+saflufenacil, compound of formula (I)+sethoxydim, compound of formula (I)+siduron, compound of formula (I)+simazine, compound of formula (I)+simetryn, compound of formula (I)+SMA, compound of formula (I)+sodium arsenite, compound of formula (I)+sodium azide, compound of formula (I)+sodium chlorate, compound of formula (I)+sulcotrione, compound of formula (I)+sulfentrazone, compound of formula (I)+sulfometuron, compound of formula (I)+sulfometuron-methyl, compound of formula (I)+sulfosate, compound of formula (I)+sulfosulfuron, compound of formula (I)+sulfuric acid, compound of formula (I)+tar oils, compound of formula (I)+2,3,6-TBA, compound of formula (I)+TCA, compound of formula (I)+TCA-sodium, formula (I)+tebutam, compound of formula (I)+tebuthiuron, formula (I)+tefuryltrione, compound of formula 1+tembotrione, compound of formula (I)+tepraloxydim, compound of formula (I)+terbacil, compound of formula (I)+terbumeton, compound of formula (I)+terbuthylazine, compound of formula (I)+terbutryn, compound of formula (I)+thenylchlor, compound of formula (I)+thiazafluoron, compound of formula (I)+thiazopyr, compound of formula (I)+thifensulfuron, compound of formula (I)+thiencarbazone, compound of formula (I)+thifensulfuron-methyl, compound of formula (I)+thiobencarb, compound of formula (I)+tiocarbazil, compound of formula (I)+topramezone, compound of formula (I)+tralkoxydim, a compound of formula (I) and triafamone compound of formula (I)+tri-allate, compound of formula (I)+triasulfuron, compound of formula (I)+triaziflam, compound of formula (I)+tribenuron, compound of formula (I)+tribenuron-methyl, compound of formula (I)+tricamba, compound of formula (I)+triclopyr, compound of formula (I)+trietazine, compound of formula (I)+trifloxysulfuron, compound of formula (I)+trifloxysulfuron-sodium, compound of formula (I)+trifluralin, compound of formula (I)+triflusulfuron, compound of formula (I)+triflusulfuron-methyl, compound of formula (I)+trifop, compound of formula (I)+trifop-methyl, compound of formula (I)+trihydroxytriazine, compound of formula (I)+trinexapac-ethyl, compound of formula (I)+tritosulfuron, compound of formula (I)+[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester (CAS RN 353292-31-6), compound of formula (I)+4-hydroxy-3-[[2-[(2-methoxyethoxy)methyl]-6-(trifluoromethyl)-3-pyridinyl]carbonyl]-bicyclo[3.2.1]oct-3-en-2-one (CAS RN 352010-68-5), compound of formula (I)+4-amino-3-chloro-6-(4-chloro-2-fluoro-3-methoxyphenyl)-2-pyridinecarboxylic acid (CAS RN 943832-60-8), and compound of formula (I)+4-hydroxy-3-[[2-(3-methoxypropyl)-6-(difluoromethyl)-3-pyridinyl]carbonyl]-bicyclo[3.2.1]oct-3-en-2-one.

Whilst two-way mixtures of a compound of formula (I) and another herbicide are explicitly disclosed above, the skilled man will appreciate that the invention extends to three-way, and further multiple combinations comprising the above two-way mixtures.

In preferred embodiments a compound of formula (I) is combined with an acetolactate synthase inhibitor, (e.g. one or more of florasulam, flumetsulam, metsulfuron, nicosulfuron, prosulfuron, thifensulfuron, tribenuron, triasulfuron, flucarbazone, flupyrsulfuron, iodosulfuron, mesosulfuron, primisulfuron, primisulfuron-methyl, propoxicarbazone, rimsulfuron, sulfosulfuron, pyroxsulam and tritosulfuron, as well as salts or esters thereof), a synthetic auxin herbicide (e.g. one or more of aminocyclopyrachlor, aminopyralid, clopyralid, 2,4-D, 2,4-DB, dicamba, dichlorprop, fluoroxypyr, MCPA, MCPB, mecoprop, mecoprop-P and 4-amino-3-chloro-6-(4-chloro-2-fluoro-3-methoxyphenyl)-2-pyridinecarboxylic acid (CAS RN 943832-60-8)), an ACCase-inhibiting herbicide (e.g. one or more of phenylpyrazolin; pinoxaden; an aryloxyphenoxypropionic herbicide such as clodinafop, cyhalofop, diclofop, fenoxaprop, fluazifop, haloxyfop, quizalofop, trifop and mixtures thereof, as well as the isomers thereof, for example, fenoxaprop-P, fluazifop-P, haloxyfop-P, quizalofop-P; and a cyclohexanedione herbicide such as alloxydim; butroxydim, clethodim, cycloxydim, profoxydim, sethoxydim, tepraloxydim and tralkoxydim, as well as salts or esters thereof), an auxin transport inhibitor such as semicarbazone (e.g. diflufenzopyr, in particular the sodium salt), or phthalamate compound (e.g. naptalam), an HPPD inhibiting herbicide (e.g. mesotrione, topramezone, tembotrione), a glutamine synthetase inhibitor such as glufosinate or glufosinate-ammonium and/or an EPSPS inhibitor such as glyphosate.

Particularly preferred mixture partners for compounds of formula (I) are: florasulam, flumetsulam, iodosulfuron-methyl-sodium, mesosulfuron-methyl, metsulfuron-methyl, nicosulfuron, primisulfuron-methyl, prosulfuron, rimsulfuron, thifensulfuron, triasulfuron, tribenuron-methyl or pyroxsulam; dicamba, fluoroxypyr, MCPA, mecoprop or mecoprop-P; clodinafop-propargyl, cyhalofop-butyl, diclofop-methyl, fenoxaprop-ethyl, fenoxaprop-P-ethyl, fluazifop-butyl, fluazifop-P-butyl, haloxyfop-methyl, haloxyfop-P-methyl, pinoxaden, propaquizafop, quizalofop-ethyl, quizalofop-P-ethyl, tralkoxydim, trifop-methyl, diflufenzopyr-Na, mesotrione, tembotrione, topramezone, naptalam, glufosinate and glyphosate.

For the avoidance of doubt, even if not explicitly stated above, the mixing partners of the compound of formula (I) may also be in the form of any suitable agrochemically acceptable ester or salt, as mentioned e.g. in The Pesticide Manual, Thirteenth Edition, British Crop Protection Council, 2003.

The mixing ratio of the compound of formula (I) to the mixing partner is preferably from 1:100 to 1000:1.

The mixtures can advantageously be used in the above-mentioned formulations (in which case “active ingredient” relates to the respective mixture of compound of formula (I) with the mixing partner).

The compounds of formula (I) according to the invention can also be used in combination with one or more safeners. Likewise, mixtures of a compound of formula (I) according to the invention with one or more further active ingredients, in particular with one or more further herbicides, can also be used in combination with one or more safeners. Where a compound of formula (I) is combined with a safener, the following combinations of the compound of formula (I) and the safener are particularly preferred. Compound of formula (I)+AD 67 (MON 4660), compound of formula (I)+benoxacor, compound of formula (I)+cloquintocet-mexyl, compound of formula (I)+cyometrinil and a compound of formula (I)+the corresponding (Z) isomer of cyometrinil, compound of formula (I)+cyprosulfamide (CAS RN 221667-31-8), compound of formula (I)+dichlormid, compound of formula (I)+fenchlorazole-ethyl, compound of formula (I)+fenclorim, compound of formula (I)+flurazole, compound of formula (I)+fluxofenim, compound of formula (I)+furilazole and a compound of formula (I)+the corresponding R isomer or furilazome, compound of formula (I)+isoxadifen-ethyl, compound of formula (I)+mefenpyr-diethyl, compound of formula (I)+oxabetrinil, compound of formula (I)+naphthalic anhydride (CAS RN 81-84-5), compound of formula (I)+N-isopropyl-4-(2-methoxy-benzoylsulfamoyl)-benzamide (CAS RN 221668-34-4) and a compound of formula (I)+N-(2-methoxybenzoyl)-4-[(methylaminocarbonyl)amino]benzenesulfonamide.

Particularly preferred safeners for use in the invention are cloquintocet-mexyl, cyprosulfamide, fenchlorazole-ethyl, mefenpyr-diethyl and N-(2-methoxybenzoyl)-4-[(methylaminocarbonyl)amino]benzenesulfonamide. In particular, the present invention provides a composition comprising any one of compounds 1-1 to 1-192, 2-2 to 2-192, 3-2 to 3-192, 4-2 to 4-192, 5-2 to 5-192, 6-2 to 6-192, 7-2 to 7-192, 8-2 to 8-192, 9-2 to 9-192, 10-2 to 10-192, 11-2 to 11-192, 12-2 to 12-192, 13-2 to 13-192, 14-2 to 14-192, 15-2 to 15-192, 16-2 to 16-192, 17-2 to 17-192, 18-2 to 18-192, 19-2 to 19-192, 20-2 to 20-192, 21-2 to 21-192, 22-2 to 22-192, 23-2 to 23-192, 24-2 to 24-192, 25-2 to 25-192, 26-2 to 26-192, 27-2 to 27-192, 28-2 to 28-192, 29-2 to 29-192, 30-2 to 30-192, 31-2 to 31-192, 32-2 to 32-192 and 33-2 to 33-192 with cloquintocet-mexyl.

In particular, the present invention provides a composition comprising any one of compounds 1-1 to 1-192, 2-2 to 2-192, 3-2 to 3-192, 4-2 to 4-192, 5-2 to 5-192, 6-2 to 6-192, 7-2 to 7-192, 8-2 to 8-192, 9-2 to 9-192, 10-2 to 10-192, 11-2 to 11-192, 12-2 to 12-192, 13-2 to 13-192, 14-2 to 14-192, 15-2 to 15-192, 16-2 to 16-192, 17-2 to 17-192, 18-2 to 18-192, 19-2 to 19-192, 20-2 to 20-192, 21-2 to 21-192, 22-2 to 22-192, 23-2 to 23-192, 24-2 to 24-192, 25-2 to 25-192, 26-2 to 26-192, 27-2 to 27-192, 28-2 to 28-192, 29-2 to 29-192, 30-2 to 30-192, 31-2 to 31-192, 32-2 to 32-192 and 33-2 to 33-192 with cyprosulfamide.

In particular, the present invention provides a composition comprising any one of compounds 1-1 to 1-192, 2-2 to 2-192, 3-2 to 3-192, 4-2 to 4-192, 5-2 to 5-192, 6-2 to 6-192, 7-2 to 7-192, 8-2 to 8-192, 9-2 to 9-192, 10-2 to 10-192, 11-2 to 11-192, 12-2 to 12-192, 13-2 to 13-192, 14-2 to 14-192, 15-2 to 15-192, 16-2 to 16-192, 17-2 to 17-192, 18-2 to 18-192, 19-2 to 19-192, 20-2 to 20-192, 21-2 to 21-192, 22-2 to 22-192, 23-2 to 23-192, 24-2 to 24-192, 25-2 to 25-192, 26-2 to 26-192, 27-2 to 27-192, 28-2 to 28-192, 29-2 to 29-192, 30-2 to 30-192, 31-2 to 31-192, 32-2 to 32-192 and 33-2 to 33-192 with fenchlorazole-ethyl.

In particular, the present invention provides a composition comprising any one of compounds 1-1 to 1-192, 2-2 to 2-192, 3-2 to 3-192, 4-2 to 4-192, 5-2 to 5-192, 6-2 to 6-192, 7-2 to 7-192, 8-2 to 8-192, 9-2 to 9-192, 10-2 to 10-192, 11-2 to 11-192, 12-2 to 12-192, 13-2 to 13-192, 14-2 to 14-192, 15-2 to 15-192, 16-2 to 16-192, 17-2 to 17-192, 18-2 to 18-192, 19-2 to 19-192, 20-2 to 20-192, 21-2 to 21-192, 22-2 to 22-192, 23-2 to 23-192, 24-2 to 24-192, 25-2 to 25-192, 26-2 to 26-192, 27-2 to 27-192, 28-2 to 28-192, 29-2 to 29-192, 30-2 to 30-192, 31-2 to 31-192, 32-2 to 32-192 and 33-2 to 33-192 with mefenpyr-diethyl.

In particular, the present invention provides a composition comprising any one of compounds 1-1 to 1-192, 2-2 to 2-192, 3-2 to 3-192, 4-2 to 4-192, 5-2 to 5-192, 6-2 to 6-192, 7-2 to 7-192, 8-2 to 8-192, 9-2 to 9-192, 10-2 to 10-192, 11-2 to 11-192, 12-2 to 12-192, 13-2 to 13-192, 14-2 to 14-192, 15-2 to 15-192, 16-2 to 16-192, 17-2 to 17-192, 18-2 to 18-192, 19-2 to 19-192, 20-2 to 20-192, 21-2 to 21-192, 22-2 to 22-192, 23-2 to 23-192, 24-2 to 24-192, 25-2 to 25-192, 26-2 to 26-192, 27-2 to 27-192, 28-2 to 28-192, 29-2 to 29-192, 30-2 to 30-192, 31-2 to 31-192, 32-2 to 32-192 and 33-2 to 33-192 with N-(2-methoxybenzoyl)-4-[(methylaminocarbonyl)amino]benzenesulfonamide.

The safeners of the compound of formula (I) may also be in the form of esters or salts, as mentioned e.g. in The Pesticide Manual, 13^{th} Edition supra. The reference to cloquintocet-mexyl also applies to a lithium, sodium, potassium, calcium, magnesium, aluminium, iron, ammonium, quaternary ammonium, sulfonium or phosphonium salt thereof as disclosed in WO02/34048, and the reference to fenchlorazole-ethyl also applies to fenchlorazole, etc.

Preferably the mixing ratio of compound of formula (I) to safener is from 100:1 to 1:10, especially from 20:1 to 1:1.

The mixtures can advantageously be used in the above-mentioned formulations (in which case “active ingredient” relates to the respective mixture of compound of formula (I) with the safener).

Preferred mixtures of a compound of formula (I) with further herbicides and safeners include: a compound of formula (I)+pinoxaden+cloquintocet-mexyl, a compound of formula (I)+clodinafop+cloquintocet-mexyl, and a compound of formula (I)+clodinafop-propargyl+cloquintocet-mexyl, a compound of formula (I)+glyphosate+cyprosulfamide (CAS RN 221667-31-8), a compound of formula (I)+glyphosate+N-(2-methoxybenzoyl)-4-[(methylaminocarbonyl)amino]benzenesulfonamide.

Various aspects and embodiments of the present invention will now be illustrated in more detail by way of example. It will be appreciated that modification of detail may be made without departing from the scope of the invention.

For the avoidance of doubt, where a literary reference, patent application, or patent, is cited within the text of this application, the entire text of said citation is herein incorporated by reference.

### EXAMPLES

### Example 1

### Synthesis of 3-bromo-6-chloro-pyridine-2-carboxylic acid methyl ester

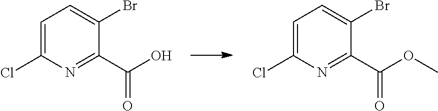

3-Bromo-6-chloro-pyridine-2-carboxylic acid (50.00 g, 211.5 mmol) was dissolved in methanol (235 mL), and concentrated sulphuric acid (5.6 mL) was added. The resulting reaction mixture was heated to reflux for 28 h. The reaction mixture was cooled to room temperature, and the resulting precipitate was recrystallized from methanol to give 3-bromo-6-chloro-pyridine-2-carboxylic acid methyl ester (54.38 g, quantitative) as a solid. Characterising data for the compound are as follows: ¹H NMR (400 MHz, CDCl₃) δ ppm 7.94 (d, 1H), 7.34 (d, 1H) and 4.01 (s, 3H).

### Example 2

### Synthesis of 3-bromo-4,6-dichloro-pyridine-2-carboxylic acid methyl ester

### 2.1 Preparation of 3-bromo-6-chloro-pyridine-2-carboxylic acid N-oxide

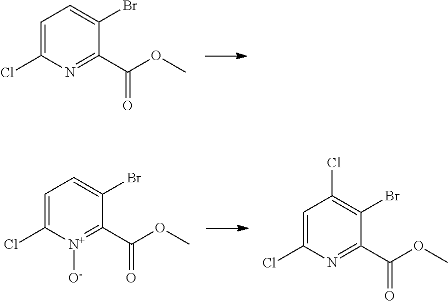

Urea hydrogen peroxide (65.32 g, 347.2 mmol) was added portionwise to a solution of trifluoroacetic anhydride (145.8 g, 694.4 mmol, 96.5 mL) in dichloromethane (577 mL) at 0° C. 3-Bromo-6-chloro-pyridine-2-carboxylic acid methyl ester (27.18 g, 108.5 mmol) was added to the mixture portionwise and the reaction was stirred at room temperature for 19 h. The reaction was quenched by the addition of water, and the organic layer was washed with water and saturated aqueous K₂CO₃. The organic layer was dried (MgSO₄) and concentrated in vacuo to give 3-bromo-6-chloro-pyridine-2-carboxylic acid N-oxide as a yellow oil. Characterising data for the compound are as follows: ¹H NMR (400 MHz, CD₃OD) δ ppm 7.77-7.75 (m, 2H) and 4.01 (s, 3H).

### 2.2 Preparation of 3-bromo-4,6-dichloro-pyridine-2-carboxylic acid methyl ester

3-Bromo-6-chloro-pyridine-2-carboxylic acid N-oxide was dissolved in POCl₃ (166 g, 1.085 mol, 101 mL) and stirred at room temperature for 2 h, then at reflux for 3 h. The reaction mixture was concentrated in vacuo, and purified by silica gel chromatography (gradient elution: 0-100% EtOAc in iso-hexane) followed by reverse phased silica gel chromatography (gradient elution: 0-100% MeOH in water) to give 3-bromo-4,6-dichloro-pyridine-2-carboxylic acid methyl ester (9.45 g, 31%) as a solid. Characterising data for the compound are as follows: ¹H NMR (400 MHz, CDCl₃) S ppm 7.57 (s, 1H) and 4.01 (s, 3H).

### Example 3

### Synthesis of 4-amino-3-bromo-6-chloro-pyridine-2-carboxylic acid methyl ester

### 3.1 Preparation of 4-azido-3-bromo-6-chloro-pyridine-2-carboxylic acid methyl ester

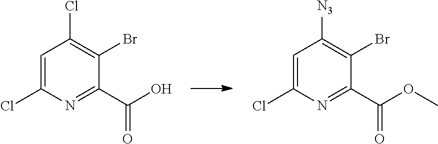

3-Bromo-4,6-dichloro-pyridine-2-carboxylic acid methyl ester (4.036 g, 14.16 mmol) and sodium azide (1.105 g, 17.00 mmol) was dissolved in DMF (7.1 mL) and heated to 60° C. After 2 h, the reaction mixture was cooled to room temperature and poured on to water. The aqueous layer was extracted with dichloromethane, the combined organic extracts were dried (MgSO₄), and concentrated in vacuo. The resulting residue was purified by silica gel chromatography (gradient elution: 0-100% EtOAc in iso-hexane) to give 4-azido-3-bromo-6-chloro-pyridine-2-carboxylic acid methyl ester (3.999 g, 97%) as a yellow solid. Characterising data for the compound are as follows: ¹H NMR (400 MHz, CDCl₃) δ ppm 7.19 (s, 1H) and 4.00 (s, 3H).

### 3.2 Preparation of 4-amino-3-bromo-6-chloro-pyridine-2-carboxylic acid methyl ester

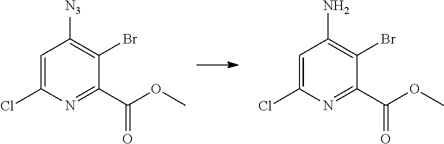

Sodium borohydride (0.588 g, 15.55 mmol) was added portionwise to a solution of 4-azido-3-bromo-6-chloro-pyridine-2-carboxylic acid methyl ester (2.266 g, 7.78 mmol) in methanol (16 mL) at 0° C. After 30 min, the reaction mixture was poured on to water, and the aqueous layer was extracted successively with dichloromethane and EtOAc. The combined organic layers were dried (MgSO₄), concentrated in vacuo and purified by silica gel chromatography (gradient elution: 0-100% EtOAc in dichloromethane) to give 4-amino-3-bromo-6-chloro-pyridine-2-carboxylic acid methyl ester (1.463 g, 71%) as a solid. Characterising data for the compound are as follows: ¹H NMR (400 MHz, CDCl₃) δ ppm 6.72 (s, 1H), 5.00 (br s, 2H) and 3.98 (s, 3H).

### Example 4

### Alternative Synthesis of 4-amino-3-bromo-6-chloro-pyridine-2-carboxylic acid methyl ester

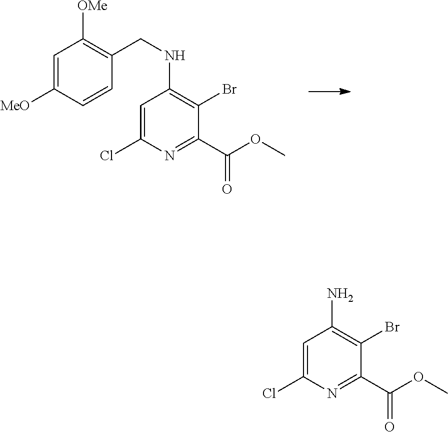 [SMILES: COC(=O)c1nc(Cl)cc(NCc2ccc(OC)cc2OC)c1Br ; COC(=O)c1nc(Cl)cc(N)c1Br]

A solution of 3-bromo-6-chloro-4-[(2,4-dimethoxyphenyl)methylamino]-pyridine-2-carboxylic acid methyl ester (18.0 g, 43.4 mmol; prepared as described in example 5) in methanolic hydrogen chloride (1.25 M; 250 ml) was stirred at 48° C. for 3 hours then cooled and filtered. The filtrate was concentrated in vacuo and the residue partitioned between ethyl aceate and saturated aqueous sodium bicarbonate. The aqueous phase was extracted with ethyl acetate and the combined organic phases washed with saturated aqueous sodium bicarbonate, water and brine, dried (MgSO₄) and concentrated in vacuo to provide 4-amino-3-bromo-6-chloro-pyridine-2-carboxylic acid methyl ester (9.3 g, 80%) as a beige solid. Characterising data for the compound are as follows: ¹H NMR (400 MHz, CDCl₃) δ ppm 6.71 (s, 1H), 5.00 (br s, 2H) and 3.98 (s, 3H).

### Example 5

### Synthesis of 3-bromo-6-chloro-4-cyclopropylamino-pyridine-2-carboxylic acid methyl ester

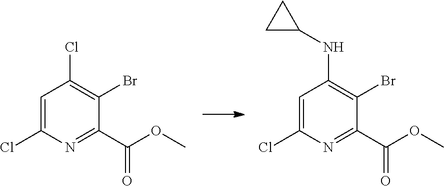 [SMILES: COC(=O)c1nc(Cl)cc(Cl)c1Br ; COC(=O)c1nc(Cl)cc(NC2CC2)c1Br]

A solution of 3-bromo-4,6-dichloro-pyridine-2-carboxylic acid methyl ester (0.545 g, 1.91 mmol) in N-methylpyrrolidone (2 mL) was added to a mixture of cyclopropylamine (109 mg, 1.91 mmol) and diisopropyethylamine (0.66 ml, 3.82 mmol) and the resulting mixture heated at 80° C. for 20 h. The reaction mixture was cooled to room temperature, water (2 ml) and dichloromethane (2 ml) added and the mixture stirred for 5 min. The phases were separated and the organic phase concentrated in vacuo. The residue was purified by reverse phase preparative HPLC, using FractionLynx (X Bridge column, ammonium acetate buffer) to give 3-bromo-6-chloro-4-cyclopropylamino-pyridine-2-carboxylic acid methyl ester, which was used directly in the next reaction.

Other compounds made using this general method are listed in Table 2 below.

TABLE 2
Compounds made according to the general method described in Example 5.
Characteristic data is melting point (° C.) or ¹H nmr data (400 MHz, CDCl₃) δH ppm.

| Name | Structure | Characteristic Data |
|---|---|---|
| 3-Bromo-6- chloro-4-(2- hydroxypropyl- amino)-pyridine- 2-carboxylic acid methyl ester | 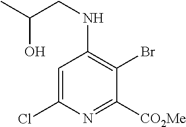 [SMILES: COC(=O)c1nc(Cl)cc(NCC(C)O)c1Br] | Used without characterisation |
| 3-Bromo-6- chloro-4- cyclopentyl- amino-pyridine-2- carboxylic acid methyl ester | 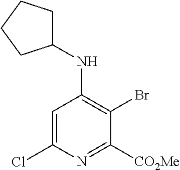 [SMILES: COC(=O)c1nc(Cl)cc(NC2CCCC2)c1Br] | Used without characterisation |
| 3-Bromo-6- chloro-4- pyrrolidin-1-yl- pyridine-2- carboxylic acid methyl ester | 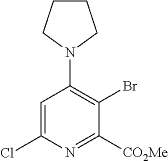 [SMILES: COC(=O)c1nc(Cl)cc(N2CCCC2)c1Br] | Used without characterisation |
| 3-Bromo-6- chloro-4-[(2,4- dimethoxyphenyl) methylamino]- pyridine-2- carboxylic acid methyl ester | 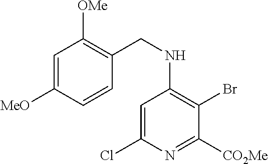 [SMILES: COC(=O)c1nc(Cl)cc(NCc2ccc(OC)cc2OC)c1Br] | 87-88 |

### Example 6

### Synthesis of 4-amino-6-chloro-3-ethenyl-pyridine-2-carboxylic acid methyl ester (Compound 2-4)

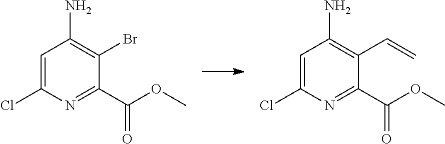

A mixture of 4-amino-3-bromo-6-chloro-pyridine-2-carboxylic acid methyl ester (186 mg, 0.70 mmol), tributyl(vinyl)tin (222 mg, 0.70 mmol, 0.205 mL) and bis(triphenylphosphine)palladium(II) dichloride (39 mg, 0.06 mmol) in DMF (3.5 mL) was heated to 150° C. under microwave irradiation for 30 min. More tributyl(vinyl)tin (33 mg, 0.10 mmol, 0.03 mL) and bis(triphenylphosphine)palladium(II) dichloride (5 mg, 0.007 mmol) were added to the mixture, and the reaction mixture was heated again to 150° C. under microwave irradiation for 30 min. The reaction mixture was filtered through Celite® and the resulting solution was purified by reverse phase preparative HPLC, using FractionLynx (X Bridge column, ammonium acetate buffer) to give 4-amino-6-chloro-3-ethenyl-pyridine-2-carboxylic acid methyl ester (71 mg, 48%) as a gum. Characterising data for the compound are as follows: ¹H NMR (400 MHz, CD₃OD) δ ppm 6.70 (s, 1H), 6.64 (dd, 1H), 5.53 (dd, 1H), 5.42 (dd, 1H), 4.59 (br s, 2H) and 3.83 (s, 3H).

Other compounds made using this general method are listed in Table 3 below.

TABLE 3
Compounds made according to the general method described in Example 6. Characteristic
data is melting point (° C.) or ¹H nmr data (400 MHz, CDCl₃) δH ppm.

| | | | Characteristic |
|---|---|---|---|
| Compound No. | Name | Structure | data |
| 2-5 (Z isomer) | (Z)-4-Amino-6- chloro-3-(prop- 1-enyl)- pyridine-2- carboxylic acid methyl ester | 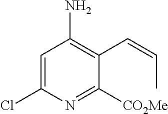 | 94-97 |
| 23-4 | 6-Chloro-4- cyclopropylamino- 3-ethenyl- pyridine-2- carboxylic acid methyl ester | 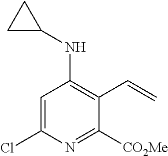 [SMILES: C=Cc1c(NC2CC2)cc(Cl)nc1C(=O)OC] | 6.96 (s, 1H), 6.67 (m, 1H), 5.59 (dd, 1H), 5.37 (dd, 1H), 5.30 (br. s, 1H), 3.87 (s, 3H), 2.42 (m, 1H), 0.86 (m, 2H), 0.55 (m, 2H) |
| 21-4 | 6-Chloro-3- ethenyl-4-(2- hydroxypropylamino)- pyridine- 2-carboxylic acid methyl ester | 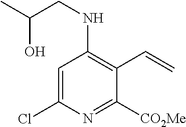 [SMILES: C=Cc1c(NCC(C)O)cc(Cl)nc1C(=O)OC] | 6.74 (m, 1H), 6.57 (s, 1H), 5.67 (dd, 1H), 5.47 (dd, 1H), 5.45 (br. s, 1H), 4.09 (m, 1H), 3.90 (s, 3H), 3.25 (m, 1H), 3.05 (m, 1H), 1.62 (d, 1H), 1.31 (d, 3H) |
| 27-4 | 6-Chloro-4- cyclopentylamino- 3-ethenyl- pyridine-2- carboxylic acid methyl ester | 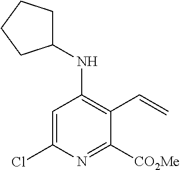 [SMILES: C=Cc1c(NC2CCCC2)cc(Cl)nc1C(=O)OC] | 6.69 (m, 1H), 6.56 (s, 1H), 5.62 (dd, 1H), 5.40 (dd, 1H), 4.96 (m, 1H), 3.88 (s, 3H), 3.75 (m, 1H), 2.04 (m, 2H), 1.70 (m, 4H), 1.48 (m, 2H) |
| 31-4 | 6-Chloro-3- ethenyl-4- pyrrolidin-1-yl- pyridine-2- carboxylic acid methyl ester | 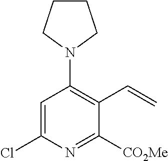 [SMILES: C=Cc1c(N2CCCC2)cc(Cl)nc1C(=O)OC] | 6.98 (m, 1H), 6.50 (s, 1H), 5.40 (dd, 1H), 5.19 (dd, 1H), 3.85 (s, 3H), 3.40 (m, 4H), 1.95 (m, 4H) |

### Example 7

### Synthesis of 4-amino-6-chloro-3-methyl-pyridine-2-carboxylic acid methyl ester (Compound 2-1)

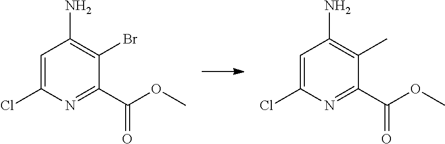

A mixture of 4-amino-3-bromo-6-chloro-pyridine-2-carboxylic acid methyl ester (186 mg, 0.70 mmol), tetramethyltin (125 mg, 0.70 mmol, 0.097 mL) and bis(triphenylphosphine)palladium(II) dichloride (39 mg, 0.06 mmol) in DMF (3.5 mL) was heated to 150° C. under microwave irradiation for 30 min. The reaction mixture was filtered through Celite® and the resulting solution was purified by reverse phase preparative HPLC, using FractionLynx (X Bridge column, ammonium acetate buffer) to give 4-amino-6-chloro-3-methyl-pyridine-2-carboxylic acid methyl ester (74 mg, 53%) as a solid. Characterising data for the compound are as follows: ¹H NMR (400 MHz, CD₃OD) δ ppm 6.67 (s, 1H), 4.59 (br s, 2H), 3.89 (s, 3H) and 2.13 (s, 3H).

### Example 8

### Synthesis of 4-amino-6-chloro-3-(2-methyl-prop-2-enyl)-pyridine-2-carboxylic acid methyl ester (Compound 2-8)

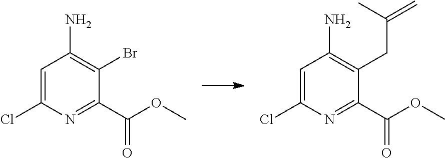 [SMILES: COC(=O)c1nc(Cl)cc(N)c1Br ; C=C(C)Cc1c(N)cc(Cl)nc1C(=O)OC]

A mixture of 4-amino-3-bromo-6-chloro-pyridine-2-carboxylic acid methyl ester (186 mg, 0.70 mmol), methallyltri-n-butylstannane (242 mg, 0.70 mmol, 0.162 mL) and bis(triphenylphosphine)palladium(II) dichloride (39 mg, 0.06 mmol) in DMF (3.5 mL) was heated to 150° C. under microwave irradiation for 30 min. More methallyltri-n-butylstannane (45 mg, 0.13 mmol, 0.03 mL) and bis(triphenylphosphine)palladium(II) dichloride (5 mg, 0.007 mmol) were added to the mixture, and the reaction mixture was heated again to 150° C. under microwave irradiation for 30 min. The reaction mixture was filtered through Celite® and the resulting solution was purified by reverse phase preparative HPLC, using FractionLynx (X Bridge column, ammonium acetate buffer) to give 4-amino-6-chloro-3-(2-methyl-prop-2-enyl)-pyridine-2-carboxylic acid methyl ester (71 mg, 42%) as a gum. Characterising data for the compound are as follows: ¹H NMR (400 MHz, CD₃OD) δ ppm 6.70 (s, 1H), 4.79-4.77 (m, 1H), 4.59 (br s, 2H), 4.52-4.50 (m, 1H), 3.85 (s, 3H), 3.39-3.37 (m, 2H) and 1.74 (s, 3H).

### Example 9

### Synthesis of 4-amino-6-chloro-3-(prop-2-enyl)-pyridine-2-carboxylic acid methyl ester (Compound 2-7)

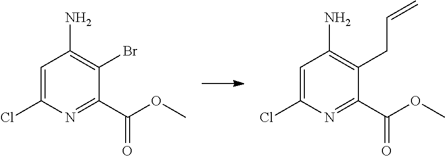

A mixture of 4-amino-3-bromo-6-chloro-pyridine-2-carboxylic acid methyl ester (159 mg, 0.60 mmol), allyltri-n-butylstannane (219 mg, 0.66 mmol, 0.203 mL) and bis(triphenylphosphine)palladium(II) dichloride (42 mg, 0.06 mmol) in DMF (3.0 mL) was heated to 150° C. under microwave irradiation for 60 min. The reaction mixture was filtered through Celite® and the resulting solution was purified by reverse phase preparative HPLC, using FractionLynx (X Bridge column, ammonium acetate buffer) to give 4-amino-6-chloro-3-(prop-2-enyl)-pyridine-2-carboxylic acid methyl ester (53 mg, 39%) as a solid. Characterising data for the compound are as follows: ¹H NMR (400 MHz, d₆-DMSO) δ ppm 6.65 (s, 1H), 6.50 (br s, 2H), 5.83-5.72 (m, 1H), 5.04-4.95 (m, 2H), 3.79 (s, 3H) and 3.29 (d, 2H).

### Example 10

### Synthesis of 4-amino-6-chloro-3-trifluoromethyl-pyridine-2-carboxylic acid methyl ester (Compound 2-2)

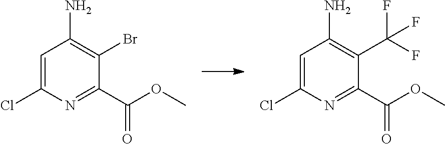 [SMILES: COC(=O)c1nc(Cl)cc(N)c1Br ; COC(=O)c1nc(Cl)cc(N)c1C(F)(F)F]

A mixture of 4-amino-3-bromo-6-chloro-pyridine-2-carboxylic acid methyl ester (199 mg, 0.75 mmol), methyl 2,2-difluoro-2-(fluorosulfonyl)acetate (158 mg, 0.83 mmol, 0.105 mL) and copper (I) iodide (43 mg, 0.23 mmol) in DMF (3.0 mL) was heated to 150° C. under microwave irradiation for 30 min. The reaction mixture was filtered through Celite® and the resulting solution was purified by reverse phase preparative HPLC, using FractionLynx (X Bridge column, ammonium acetate buffer) to give 4-amino-6-chloro-3-trifluoromethyl-pyridine-2-carboxylic acid methyl ester (10.6 mg, 6%) as a gum. Characterising data for the compound are as follows: ¹H NMR (400 MHz, CD₃OD) δ ppm 6.83 (s, 1H), 4.59 (br s, 2H) and 3.89 (s, 3H).

### Example 11

### Synthesis of 4-amino-6-chloro-3-ethenyl-5-fluoro-pyridine-2-carboxylic acid methyl ester (Compound 2-12)

### 11.1 Preparation of 4-amino-3-bromo-6-chloro-5-fluoro-pyridine-2-carboxylic acid methyl ester

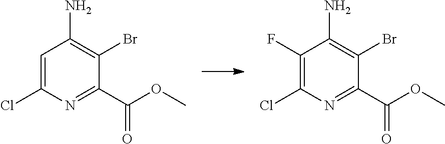

A mixture of 4-amino-3-bromo-6-chloro-pyridine-2-carboxylic acid methyl ester (864 mg, 3.25 mmol) and Selectfluor® (1.268 g, 3.58 mmol) in acetonitrile (20 mL) was heated to reflux for 150 min. More Selectfluor® (1.15 g, 3.25 mmol) was added, and the reaction mixture was further refluxed for 100 min. The reaction mixture was poured on to water and the aqueous layer was extracted with dichloromethane. The combined organic layers were dried (MgSO₄), concentrated in vacuo and purified by silica gel chromatography (gradient elution: 0-40% EtOAc in iso-hexane) to give impure 4-amino-3-bromo-6-chloro-5-fluoro-pyridine-2-carboxylic acid methyl ester (559 mg).

### 11.2 Preparation of 4-amino-6-chloro-3-ethenyl-5-fluoro-pyridine-2-carboxylic acid methyl ester

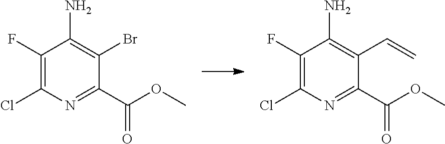

A mixture of crude 4-amino-3-bromo-6-chloro-5-fluoro-pyridine-2-carboxylic acid methyl ester (559 mg), tributyl(vinyl)tin (360 mg, 1.13 mmol, 0.331 mL) and bis(triphenylphosphine)palladium(II) dichloride (125 mg, 0.18 mmol) in DMF (10 mL) was heated to 150° C. under microwave irradiation for 60 min. The reaction mixture was concentrated in vacuo and purified by silica gel chromatography (gradient elution: 0-40% EtOAc in iso-hexane) followed by reverse phase preparative HPLC, using FractionLynx (X Bridge column, ammonium acetate buffer) to give 4-amino-6-chloro-3-ethenyl-5-fluoro-pyridine-2-carboxylic acid methyl ester (38 mg, 5% over two steps) as a solid. Characterising data for the compound are as follows: ¹H NMR (400 MHz, CDCl₃) δ ppm 6.88 (dd, 1H), 5.73 (dd, 1H), 5.53 (dd, 1H), 4.80 (br s, 2H) and 3.92 (s, 3H).

### Example 12

### Alternative Synthesis of 4-amino-6-chloro-3-ethenyl-5-fluoro-pyridine-2-carboxylic acid methyl ester (Compound 2-12)

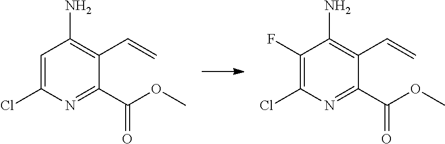

A solution of Selectfluor® (2.92 g, 8.25 mmol) in water (10 ml) was added to a solution of 4-amino-6-chloro-3-ethenyl-pyridine-2-carboxylic acid methyl ester (877 mg, 4.12 mmol) in acetonitrile (10 mL) and the resulting mixture was heated at 70° C. for 4 hours. The reaction mixture was allowed to cool, poured on to water and extracted with dichloromethane. The combined organic layers were dried (MgSO₄), concentrated in vacuo and purified by reverse phase preparative HPLC, using a FractionLynx (X Bridge column, ammonium acetate buffer) followed by silica gel chromatography (gradient elution: 0-80% EtOAc in iso-hexane) to give 4-amino-6-chloro-3-ethenyl-5-fluoro-pyridine-2-carboxylic acid methyl ester (41 mg, 4%).

### Example 13

### Synthesis of 4-amino-5,6-dichloro-3-ethenyl-1-pyridine-2-carboxylic acid methyl ester (Compound 2-20)

### 13.1 Preparation of 4-amino-3-bromo-5,6-dichloro-pyridine-2-carboxylic acid methyl ester

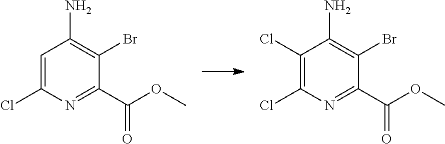

N-Chlorosuccinimide (12.6 g, 95 mmol) was added portionwise to a stirred solution of 4-amino-3-bromo-6-chloro-pyridine-2-carboxylic acid methyl ester (23.0 g, 86 mmol) in DMF (150 ml) and the resulting solution stirred at ambient temperature for 20 hours, then poured into cold water. The resulting mixture was filtered and the solid dissolved in ethyl acetate. The resulting solution was washed with water and brine, dried (MgSO₄) and concentrated in vacuo to provide 4-amino-3-bromo-5,6-dichloro-pyridine-2-carboxylic acid methyl ester (21.0 g, 81%) as a white solid. Characterising data for the compound are as follows: ¹H NMR (400 MHz, CDCl₃) δ ppm 5.50 (br s, 2H) and 3.98 (s, 3H).

### 13.2 Preparation of 4-amino-5,6-dichloro-3-ethenyl-1-pyridine-2-carboxylic acid methyl ester

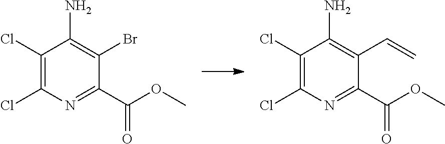

A mixture of 4-amino-3-bromo-5,6-dichloro-pyridine-2-carboxylic acid methyl ester (1.00 g, 3.33 mmol), tributyl(vinyl)tin (1.16 g, 3.67 mmol) and bis(triphenylphosphine)palladium(II) dichloride (117 mg, 0.17 mmol) in DMF (10 mL) was heated to 160° C. under microwave irradiation for 15 min. The reaction mixture was cooled, brine added and extracted with dichloromethane. The combined organic extracts were concentrated in vacuo and purified by reverse phase preparative HPLC, using a FractionLynx (X Bridge column, ammonium acetate buffer) followed by silica gel chromatography (gradient elution: 10-60% EtOAc in iso-hexane) to give 4-amino-5,6-dichloro-3-ethenyl-1-pyridine-2-carboxylic acid methyl ester (422 mg, 51%) as a white solid. Characterising data for the compound are as follows: ¹H NMR (400 MHz, CDCl₃) δ ppm 6.86 (m, 1H), 5.72 (dd, 1H), 5.53 (dd, 1H), 5.19 (br s, 2H) and 3.92 (s, 3H).

Other compounds made using this general method are listed in Table 4 below.

TABLE 4
Compounds made according to the general method
described in Example 13.

| Com- | | | ¹H NMR (400 |
|---|---|---|---|
| pound | | | MHz, CDCl₃) δ |
| No. | Name | Structure | ppm |
| 2-180 | 4-Amino-6- chloro-3- (1,2- difluoro- ethenyl)- pyridine-2- carboxylic acid methyl ester | 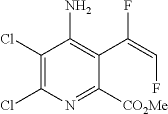 [SMILES: COC(=O)c1nc(Cl)c(Cl)c(N)c1/C(F)=C\F] | 7.40 (dd, 2H), 5.24 (br s, 2H), 3.95 (s, 3H) |

### Example 14

### Synthesis of 4-amino-5,6-dichloro-3-(1-methylethenyl)-pyridine-2-carboxylic acid methyl ester (Compound 2-22)

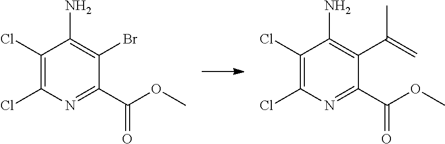 [SMILES: COC(=O)c1nc(Cl)c(Cl)c(N)c1Br ; C=C(C)c1c(C(=O)OC)nc(Cl)c(Cl)c1N]

A mixture of 4-amino-3-bromo-5,6-dichloro-pyridine-2-carboxylic acid methyl ester (200 mg, 0.67 mmol), isopropenylboronic acid pinacol ester (134 mg, 0.80 mmol), caesium fluoride (203 mg, 1.33 mmol) and tetrakis(triphenylphosphine)palladium (39 mg, 0.033 mmol) in dioxane (3 mL) and water (1 ml) was heated to 150° C. under microwave irradiation for 20 min. The reaction mixture was cooled, added to water and extracted with dichloromethane. The combined organic extracts were concentrated in vacuo and purified by silica gel chromatography (gradient elution: 0-80% EtOAc in iso-hexane) and then by reverse phase preparative HPLC, using a FractionLynx (X Bridge column, ammonium acetate buffer) followed by to give 4-amino-5,6-dichloro-3-(1-methylethenyl)-pyridine-2-carboxylic acid methyl ester as a white solid. Characterising data for the compound are as follows: ¹H NMR (400 MHz, CDCl₃) δ ppm 5.42 (m, 1H), 5.03 (br s, 2H), 4.99 (m, 1H), 3.90 (s, 3H) and 2.09 (m, 3H).

Other compounds made using this general method are listed in Table 5 below.

TABLE 5
Compounds made according to the general method described in Example 14. Characteristic
data is melting point (° C.) or ¹H nmr data (400 MHz, CDCl₃) δH ppm.

| | | | Characteristic |
|---|---|---|---|
| Compound No. | Name | Structure | Data |
| 2-180 | 4-Amino-6- chloro-3-(2- ethoxyethenyl)- pyridine-2- carboxylic acid methyl ester | 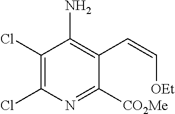 [SMILES: CCO/C=C\c1c(C(=O)OC)nc(Cl)c(Cl)c1N] | 6.37 (d, 1H), 5.49 (d, 1H), 5.23 (br s, 2H), 4.96 (q, 2H), 4.91 (s, 3H), 1.29 (t, 3H) |
| 2-5 (E isomer) | (E)-4-Amino-6- chloro-3-(prop- 1-enyl)- pyridine-2- carboxylic acid methyl ester | 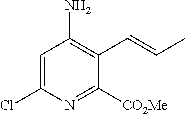 | 86-88 |
| 2-168 | 4-Amino-6- chloro-3-(4- chlorophenyl)- pyridine-2- carboxylic acid methyl ester | 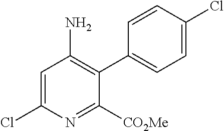 [SMILES: COC(=O)c1nc(Cl)cc(N)c1-c1ccc(Cl)cc1] | 125-127 |
| 2-165 | 4-Amino-6- chloro-3-(2- methylprop-1- enyl)-pyridine- 2-carboxylic acid methyl ester | 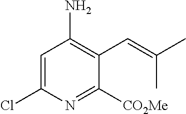 [SMILES: COC(=O)c1nc(Cl)cc(N)c1C=C(C)C] | 6.68 (s, 1H), 6.03 (m, 1H), 4.52 (br s, 2H), 3.87 (s, 3H), 1.93 (d, 3H), 1.51 (s, 3H) |

### Example 15

### Synthesis of 4-amino-6-chloro-3-formyl-pyridine-2-carboxylic acid (Compound 2-162)

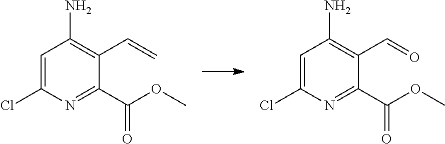

Ozone was bubbled through a stirred solution of 4-amino-6-chloro-3-ethenyl-pyridine-2-carboxylic acid methyl ester (150 mg, 1.66 mmol) in DCM (50 ml) at −78° C. for 40 mins, then dimethyl sulphide (2 ml) was added and the resulting mixture allowed to warm to ambient temperature and evaporated under reduced pressure. The residue was purified by silica gel chromatography (gradient elution: 33-50% EtOAc in iso-hexane) to provide 4-amino-6-chloro-3-formyl-pyridine-2-carboxylic acid (78 mg, 22%) as a cream solid. Characterising data for the compound are as follows: M.p. 146-148° C.; ¹H NMR (400 MHz, CDCl₃) δ ppm 10.25 (s, 1H), 6.70 (s, 1H), 4.00 (s, 3H) (amine protons not observed).

### Example 16

### Synthesis of 4-amino-6-chloro-3-difluoromethyl-pyridine-2-carboxylic acid (Compound 2-161)

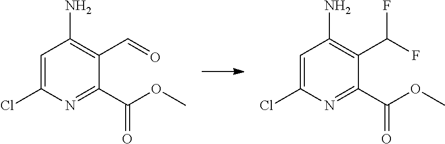

Diethylaminosulphur trifluoride (328 mg, 2.0 mmol) was added to a solution of 4-amino-6-chloro-3-formyl-pyridine-2-carboxylic acid (50 mg, 0.23 mmol) in DCM (3.5 ml) and diethyl ether (2 ml). After stirring for 40 hours further diethylaminosulphur trifluoride (160 mg, 1.0 mmol) was added and stirring continued for a further 3 days. Methanol was added and the resulting mixture concentrated in vacuo and purified by silica gel chromatography (1:2 EtOAc:iso-hexane) to provide 4-amino-6-chloro-3-difluoromethyl-pyridine-2-carboxylic acid (27 mg, 49%) as a cream solid. Characterising data for the compound are as follows: M.p. 149-151° C.; ¹H NMR (400 MHz, CDCl₃) δ ppm 7.43 (t, 1H), 6.73 (s, 1H), 5.10 (br s, 2H), 3.99 (s, 3H).

### Example 17

### Synthesis of 4-amino-6-chloro-3-ethenyl-pyridine-2-carboxylic acid (Compound 1-4)

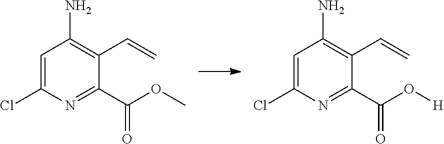

Sodium hydroxide (64 mg, 1.6 mmol) was added to a stirred solution of 4-amino-6-chloro-3-ethenyl-pyridine-2-carboxylic acid methyl ester (150 mg, 0.71 mmol) in THF (20 mL) and water (9 ml). The reaction mixture was stirred overnight at ambient temperature, then washed with ether, neutralised by the addition of 2N hydrochloric acid and extracted with ethyl acetate (3×50 ml). The combined ethyl acetate extracts were dried over magnesium sulphate, filtered and evaporated under reduced pressure to provide 4-amino-6-chloro-3-ethenyl-pyridine-2-carboxylic acid (34 mg, 24%) as a pale yellow solid. Characterising data for the compound are as follows: M.p. 141-146° C. (dec.); ¹H NMR (400 MHz, d₆-DMSO) δ ppm 6.64 (s, 1H), 6.60 (dd, 1H), 6.50 (br s, 2H), 5.49 (d, 1H), 5.44 (dd, 1H) (acid proton not observed).

Other compounds made using this general method are listed in Table 6 below.

TABLE 6
Compounds made according to the general method described in Example
17. Characteristic data is melting point (° C.) or ¹H nmr data (400 MHz,
CD₃OD) δH ppm.

| Com- | | | |
|---|---|---|---|
| pound | | | Characteristic |
| No. | Name | Structure | Data |
| 1-12 | 4-Amino-6- chloro-3- ethenyl-5- fluoro- pyridine- 2-carboxylic acid | 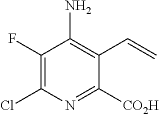 | 6.77 (m, 1H), 5.62 (dd, 1H), 5.53 (dd, 1H) (acid and amine protons not observed) |
| 1-20 | 4-Amino-5,6- dichloro-3- ethenyl- pyridine-2- carboxylic acid | 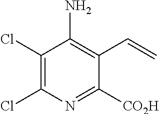 | 193-196 (dec.) |

### Example 18

### Pre-Emergence Biological Efficacy

Seeds of Alopecurus myosuroides (ALOMY), Setaria faberi (SETFA), Echinochloa crus-galli (ECHCG), Solanum nigrum (SOLNI), Amaranthus retroflexus (AMARE) and Ipomoea hederaceae (IPOHE) were sown in standard soil in pots. After cultivation for one day under controlled conditions in a glasshouse (at 24/16° C., day/night; 14 hours light; 65% humidity), the plants were sprayed with an aqueous spray solution derived from the formulation of the technical active ingredient in acetone/water (50:50) solution containing 0.5% Tween 20 (polyoxyethylene sorbitan monolaurate, CAS RN 9005-64-5) to give a final dose of 1000 g/ha of test compound.

The test plants were then grown under controlled conditions in the glasshouse (at 24/16° C., day/night; 14 hours light; 65% humidity) and watered twice daily. After 13 days the test was evaluated (100=total damage to plant; 0=no damage to plant). Results are shown below in Table 7.

TABLE 7
Percentage damage caused to weed species by compounds
of the invention when applied pre-emergence.

| Com- | | | | | | | |
|---|---|---|---|---|---|---|---|
| pound | | | | | | | |
| Num- | Rate | Species | | | | | |
| ber | (g/ha) | SOLNI | AMARE | SETFA | ALOMY | ECHCG | IPOHE |
| 1-4 | 1,000 | 100 | 100 | 100 | 90 | 90 | 100 |
| 1-12 | 1,000 | 100 | 100 | 100 | 90 | 90 | 100 |
| 1-20 | 1,000 | 100 | 100 | 90 | 90 | 90 | 100 |
| 2-1 | 1,000 | 100 | 100 | 80 | 90 | 70 | 80 |
| 2-2 | 1,000 | 80 | 80 | 0 | 20 | 10 | 90 |
| 2-4 | 1,000 | 100 | 100 | 90 | 90 | 90 | 100 |
| 2-5 | 1,000 | 100 | 90 | 100 | 90 | 70 | 100 |
| (E | | | | | | | |
| iso- | | | | | | | |
| mer) | | | | | | | |
| 2-5 | 1,000 | 100 | 100 | 100 | 90 | 70 | 90 |
| (Z | | | | | | | |
| iso- | | | | | | | |
| mer) | | | | | | | |
| 2-7 | 1,000 | 90 | 90 | 40 | 30 | 40 | 70 |
| 2-8 | 1,000 | 80 | 40 | 10 | 10 | 0 | 80 |
| 2-12 | 1,000 | 100 | 100 | 90 | 100 | 100 | 100 |
| 2-20 | 1,000 | 100 | 100 | 90 | 90 | 90 | 100 |
| 2-161 | 1,000 | 90 | 90 | 60 | 40 | 10 | 80 |
| 2-162 | 1,000 | 80 | 80 | 20 | 20 | 20 | 70 |
| 2-165 | 1,000 | 100 | 100 | 90 | 80 | 70 | 90 |
| 2-168 | 1,000 | 80 | 90 | 0 | 30 | 20 | 80 |
| 21-4 | 1,000 | 100 | 100 | 90 | 90 | 90 | 80 |
| 23-4 | 1,000 | 100 | 100 | 80 | 70 | 70 | 80 |
| 27-4 | 1,000 | 100 | 100 | 70 | 50 | 10 | 50 |
| 31-4 | 1,000 | 90 | 100 | 50 | 60 | 10 | 80 |

### Example 19

### Post-Emergence Biological Efficacy

Seeds of Alopecurus myosuroides (ALOMY), Setaria faberi (SETFA), Echinochloa crus-galli (ECHCG), Solanum nigrum (SOLNI), Amaranthus retroflexus (AMARE) and Ipomoea hederaceae (IPOHE) were sown in standard soil in pots. After cultivation for 8 days under controlled conditions in a glasshouse (at 24/16° C., day/night; 14 hours light; 65% humidity), the plants were sprayed with an aqueous spray solution derived from the formulation of the technical active ingredient in acetone/water (50:50) solution containing 0.5% Tween 20 (polyoxyethylene sorbitan monolaurate, CAS RN 9005-64-5) to give a final dose of 1000 g/ha of test compound.

The test plants were then grown on under controlled conditions in a glasshouse (at 24/16° C., day/night; 14 hours light; 65% humidity) and watered twice daily. After 13 days the test was evaluated (100=total damage to plant; 0=no damage to plant). Results are shown below in Table 8.

TABLE 8
Percentage damage caused to weed species by compounds
of the invention when applied post-emergence

| Com- | | | | | | | |
|---|---|---|---|---|---|---|---|
| pound | | | | | | | |
| Num- | Rate | Species | | | | | |
| ber | (g/ha) | SOLNI | AMARE | SETFA | ALOMY | ECHCG | IPOHE |
| 1-4 | 1,000 | 100 | 100 | 100 | 80 | 80 | 70 |
| 1-12 | 1,000 | 90 | 100 | 90 | 90 | 90 | 100 |
| 1-20 | 1,000 | 90 | 100 | 90 | 90 | 90 | 100 |
| 2-1 | 1,000 | 90 | 100 | 100 | 90 | 60 | 50 |
| 2-2 | 1,000 | 80 | 80 | 10 | 10 | 10 | 70 |
| 2-4 | 1,000 | 100 | 100 | 100 | 90 | 80 | 80 |
| 2-5 | 1,000 | 90 | 100 | 70 | 50 | 60 | 70 |
| (E | | | | | | | |
| iso- | | | | | | | |
| mer) | | | | | | | |
| 2-5 | 1,000 | 100 | 100 | 80 | 50 | 60 | 50 |
| (Z | | | | | | | |
| iso- | | | | | | | |
| mer) | | | | | | | |
| 2-7 | 1,000 | 90 | 90 | 30 | 20 | 10 | 50 |
| 2-8 | 1,000 | 80 | 70 | 10 | 10 | 0 | 50 |
| 2-12 | 1,000 | 100 | 100 | 90 | 80 | 80 | 80 |
| 2-20 | 1,000 | 100 | 100 | 90 | 90 | 90 | 100 |
| 2-161 | 1,000 | 90 | 80 | 20 | 30 | 10 | 30 |
| 2-162 | 1,000 | 90 | 80 | 10 | 10 | 10 | 70 |
| 2-165 | 1,000 | 90 | 100 | 90 | 70 | 70 | 70 |
| 2-168 | 1,000 | 80 | 100 | 20 | 20 | 10 | 70 |
| 21-4 | 1,000 | 100 | 100 | 80 | 70 | 80 | 50 |
| 23-4 | 1,000 | 100 | 100 | 80 | 60 | 30 | 70 |
| 27-4 | 1,000 | 80 | 100 | 70 | 10 | 10 | 60 |
| 31-4 | 1,000 | 90 | 90 | 50 | 20 | 20 | 70 |
| 1-4 | 1,000 | 100 | 100 | 100 | 80 | 80 | 70 |

### Example 20

### Safening on Corn

Maize seeds were sown into standard soil in pots and cultivated under controlled conditions in a glasshouse (at 24/18° C. day/night; 16 hours light; 65% humidity).

When the plants were at the vegetative stage of 3 leaves they were sprayed with an aqueous spray solution containing the components of the invention alone and where appropriate the herbicide safener (N-(2-methoxybenzoyl)-4-[(methylaminocarbonyl)amino]benzenesulfonamide). All the compounds used for the spray solution were present as an EC or SC formulation respectively. In addition a non-ionic surfactant (X-77 Spreader) was added to form a 0.2% v/v solution.

The spray solution was applied with a cabinet tracksprayer with a flat fan nozzle (Teejet 11002VS) and an application volume of 200 L/ha (at 2 bar).

The test plants were then grown on in a glasshouse under controlled conditions (at 24/18° C. day/night; 16 hours light; 65% humidity) and watered twice a day. After 7 and 17 days the test was evaluated for general crop injury (100% equals total damage to plant; 0% equals no damage to plant).

Results are shown below in Table 9.

TABLE 9
Percentage damage caused to corn by the compositions of the invention
alone and in the presence of a safener (N-(2-methoxybenzoyl)-4-
[(methylaminocarbonyl)amino]benzenesulfonamide).

| | | 7 days after application | | 17 days after application | |
|---|---|---|---|---|---|
| Compound | Rate (g | Maize cv | Maize cv | Maize cv | Maize cv |
| Number | ai/ha) | GARLAND | CLAXXON | GARLAND | CLAXXON |
| 1-4 | 200 | 60 | 60 | 65 | 50 |
| 1-4 & Safener | 200 + 200 | 40 | 40 | 10 | 10 |
| 1-12 | 200 | 60 | 60 | 50 | 50 |
| 1-12 & Safener | 200 + 200 | 30 | 40 | 20 | 20 |

Although the invention has been described with reference to preferred embodiments and examples thereof, the scope of the present invention is not limited only to those described embodiments. As will be apparent to persons skilled in the art, modifications and adaptations to the above-described invention can be made without departing from the spirit and scope of the invention, which is defined and circumscribed by the appended claims. All publications cited herein are hereby incorporated by reference in their entirety for all purposes to the same extent as if each individual publication were specifically and individually indicated to be so incorporated by reference.

1. A compound of formula (I) 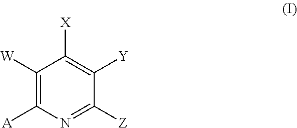
or salt or N-oxide thereof, wherein:
A is halogen, cyano, optionally substituted alkoxy, optionally substituted aryloxy, optionally substituted heteroaryloxy, optionally substituted alkylthio, optionally substituted arylthio, or optionally substituted heteroarylthio;
W is hydrogen, halogen, cyano, nitro, hydroxyl, amino, optionally substituted alkyl, optionally substituted haloalkyl, optionally substituted cycloalkyl, optionally substituted alkoxy, optionally substituted alkylamino, optionally substituted dialkylamino, optionally substituted alkylthio, optionally substituted alkylsulphinyl, optionally substituted alkylsulphonyl or optionally substituted aryl;
X is azido, nitro, optionally substituted alkoxy, optionally substituted alkylthio or —NR⁵R⁶ and
(i) R⁵ is hydrogen, optionally substituted C₁₋₄ alkyl provided said substitution does not comprise a ring system, optionally substituted C₁₋₄ haloalkyl provided said substitution does not comprise a ring system, optionally substituted C₃₋₆ cycloalkyl, C₂₋₄ alkenyl, C₂₋₄ alkynyl, —SO₂R², or —C(O)R³ and R⁶ is hydrogen, optionally substituted C₁₋₄ alkyl provided said substitution does not comprise a ring system, optionally substituted C₁₋₄ haloalkyl provided said substitution does not comprises a ring system, optionally substituted C₃₋₆ cycloalkyl, C₂₋₄ alkenyl, or C₂₋₄ alkynyl, or
(ii) R⁵ and R⁶ together form a group ═C(R⁸)OR⁹, ═C(R¹⁰)SR⁹, ═C(R¹¹)NR⁷₂, or
(iii) R⁵ and R⁶ together with the N atom to which they are attached form a 3 to 8 membered optionally substituted heterocyclyl or heteroaryl ring system, said ring system optionally containing 1 to 2 further heteroatoms independently selected from O, S and N, and R² is optionally substituted C₁₋₄ alkyl or phenyl optionally substituted by 1 to 3 groups R⁴, R³ is optionally substituted C₁₋₄ alkyl, phenyl optionally substituted by 1 to 3 groups R⁴, C₁₋₄ alkoxy, or —NR⁷₂, each R⁴ is independently halogen, C₁₋₄ alkyl, C₁₋₄ alkoxy, or C₁₋₄ alkylsulphonyl, R⁸ is hydrogen, C₁₋₄ alkyl, C₃₋₆ cycloalkyl, phenyl, C₁₋₄ alkoxy, C₁₋₄ alkylthio, or —NR⁷₂, R⁹ is C₁₋₄ alkyl, R¹⁰ is hydrogen, C₁₋₄ alkyl, C₃₋₆ cycloalkyl, phenyl, C₁₋₄ alkylthio, or —NR⁷₂, R¹¹ is hydrogen, C₁₋₄ alkyl, C₃₋₆ cycloalkyl, phenyl, or —NR⁷₂, and each R⁷ is independently hydrogen or C₁₋₄ alkyl;
Y is optionally substituted alkyl, optionally substituted haloalkyl, optionally substituted cycloalkyl, optionally substituted alkenyl, optionally substituted alkynyl, or optionally substituted aryl;
Z is —C(O)R¹², —C(S)R¹³, or —C(═NR¹⁴)R¹⁵ and R¹² is hydrogen, hydroxyl, optionally substituted alkoxy, optionally substituted alkenyloxy, optionally substituted cycloalkoxy, optionally substituted alkylthio, amino, optionally substituted alkylamino or optionally substituted dialkylamino, R¹³ is optionally substituted alkoxy, optionally substituted cycloalkoxy, optionally substituted alkylthio, amino, optionally substituted alkylamino or optionally substituted dialkylamino, R¹⁴ is hydrogen, optionally substituted alkyl, optionally substituted alkoxy, optionally substituted cycloalkoxy, amino, optionally substituted alkylamino or optionally substituted dialkylamino and R¹⁵ is hydrogen, optionally substituted alkoxy, optionally substituted cycloalkoxy, optionally substituted alkylthio, amino, optionally substituted alkylamino or optionally substituted dialkyamino.

2. A compound according to claim 1, wherein A is halogen, cyano, C₁₋₆ alkoxy optionally substituted by 1 to 3 groups R¹⁶, C₁₋₆ haloalkoxy optionally substituted by 1 to 3 groups R¹⁶, C₁₋₆ alkylthio optionally substituted by 1 to 3 groups R¹⁶, C₁₋₆ haloalkylthio optionally substituted by 1 to 3 groups R¹⁶, aryloxy optionally substituted by 1 to 3 groups R¹, heteroaryloxy optionally substituted by 1 to 3 groups R¹, arylthio optionally substituted by 1 to 3 groups R¹ or heteroarylthio optionally substituted by 1 to 3 groups R¹, each R¹ is independently halogen, cyano, nitro, hydroxyl, C₁₋₆ alkyl optionally substituted by 1 to 4 groups R¹⁶, C₁₋₆ haloalkyl optionally substituted by 1 to 4 groups R¹⁶, —OR¹⁷, —S(O)ₐR¹⁸, —C(O)R¹⁹, or —NR²⁰₂ or any two geminal groups R¹ together form a group selected from oxo, ═CR²¹₂, ═NOR²², or ═NNR²²R²³, each R¹⁶ is independently cyano, hydroxyl, C₃₋₆ cycloalkyl, —OR¹⁷, —S(O)ₐR¹⁸, —C(O)R¹⁹ or —NR²⁰₂, and
(i) each R¹⁷ is independently C₁₋₆ alkyl, C₁₋₆ haloalkyl, C₁₋₄ alkoxy(C₁₋₄)alkyl, or C₁₋₆ alkylcarbonyl,
(ii) each R¹⁸ is independently C₁₋₆ alkyl, C₁₋₆ haloalkyl, C₂₋₆ alkenyl, C₂₋₆ alkynyl, C₃₋₆ cycloalkyl, or C₁₋₆ alkylcarbonylamino and each a is, independently, 0, 1, or 2,
(iii) each R¹⁹ is independently hydrogen, hydroxyl, C₁₋₆ alkyl, C₁₋₆ haloalkyl, C₃₋₆ cycloalkyl, C₁₋₆ alkoxy, phenyl(C₁₋₆)alkoxy, C₃₋₆ cycloalkoxy, amino, C₁₋₆ alkylamino, di(C₁₋₄)alkylamino, or C₁₋₆ alkylsulphonylamino,
(iv) each R²⁰ is independently hydrogen or C₁₋₄ alkyl,
(v) each R²¹ is independently hydrogen, halogen, cyano, nitro, C₁₋₆ alkyl, C₃₋₆ cycloalkyl, C₁₋₆ alkylcarbonyl, C₁₋₆ alkoxycarbonyl, C₁₋₆ alkylsulphonyl, or aminocarbonyl,
(vi) each R²² is independently hydrogen, C₁₋₆ alkyl, or C₃₋₆ cycloalkyl and
(vii) each R²³ is independently hydrogen or C₁₋₆ alkyl.

3. A compound according to claim 2, wherein A is halogen, C₁₋₄ alkylthio optionally substituted by 1 to 3 groups R¹⁶, C₁₋₄ haloalkylthio optionally substituted by 1 to 3 groups R¹⁶ or aryloxy optionally substituted by 1 to 3 groups R¹.

4. A compound according to claim 3, wherein A is halogen or aryloxy optionally substituted by 1 to 3 groups R¹.

5. A compound according to claim 4, wherein each R¹ is independently halogen, cyano, C₁₋₂ alkyl, C₁₋₂ haloalkyl, C₁₋₂ alkoxy, C₁₋₂ haloalkoxy, or di(C₁₋₂)alkylamino.

6. A compound according to claim 1, wherein W is hydrogen, halogen, cyano, nitro, hydroxyl, amino, C₁₋₆ alkyl optionally substituted by 1 to 3 groups R³⁰, C₁₋₆ haloalkyl optionally substituted by 1 to 3 groups R³⁰, C₃₋₆ cycloalkyl optionally substituted by 1 to 3 groups R³⁰, C₁₋₆ alkoxy optionally substituted by 1 to 3 groups R³⁰, C₁₋₆ alkylamino optionally substituted by 1 to 3 groups R³⁰, di(C₁₋₆)alkylamino optionally substituted by 1 to 3 groups R³⁰, C₁₋₆ alkylthio optionally substituted by 1 to 3 groups R³⁰, C₁₋₆ alkylsulphinyl optionally substituted by 1 to 3 groups R³⁰, C₁₋₆ alkylsulphonyl optionally substituted by 1 to 3 groups R³⁰ or C₅₋₁₀ aryl optionally substituted by 1 to 3 groups R³⁰ and each R³⁰ is independently selected from halogen, hydroxyl, cyano, amino, nitro, C₁₋₆ alkylamino, di(C₁₋₆)alkylamino, C₁₋₆ alkyl, C₁₋₆ haloalkyl, C₃₋₆ cycloalkyl, C₁₋₆ alkoxy, C₁₋₆ haloalkoxy, C₁₋₆ alkylthio, C₁₋₆ alkylcarbonyl or C₁₋₆ alkoxycarbonyl.

7. A compound according to claim 6, wherein W is hydrogen, halogen, C₁₋₃ alkyl, C₁₋₃ haloalkyl, C₁₋₂ alkoxy(C₁₋₂)alkyl or cyclopropyl optionally substituted by 1 or 2 groups independently selected from halogen or C₁₋₆ alkyl.

8. A compound according to claim 7, wherein W is hydrogen, halogen, C₁₋₂ alkyl, C₁₋₂ haloalkyl, C₁₋₂ alkoxy(C₁₋₂)alkyl, or cyclopropyl.

9. A compound according to claim 1, wherein X is azido, nitro, alkoxy optionally substituted by 1 to 3 groups R³¹, alkylthio optionally substituted by 1 to 3 groups R³¹ or —NR⁵R⁶, and
(i) R⁵ is hydrogen, C₁₋₄ alkyl optionally substituted by 1 to 4 groups R²⁴, C₁₋₄ haloalkyl optionally substituted by 1 to 4 groups R²⁴, C₃₋₆ cycloalkyl optionally substituted by 1 to 4 groups R²⁴, C₂₋₄ alkenyl, C₂₋₄ alkynyl, —SO₂R², or —C(O)R³ and R⁶ is hydrogen, C₁₋₄ alkyl optionally substituted by 1 to 4 groups R²⁴, C₁₋₄ haloalkyl optionally substituted by 1 to 4 groups R²⁴, C₃₋₆ cycloalkyl optionally substituted by 1 to 4 groups R²⁴, C₂₋₄ alkenyl, or C₂₋₄ alkynyl, or
(ii) R⁵ and R⁶ together form a group ═C(R⁸)OR⁹, ═C(R¹⁰)SR⁹, ═C(R¹¹)NR⁷₂ or
(iii) R⁵ and R⁶ together with the N atom to which they are attached form a 3 to 8 membered heterocyclyl or heteroaryl ring system, said ring system optionally containing 1 to 2 further heteroatoms independently selected from O, S and N and being optionally substituted by 1 to 3 groups R³³
and each R³¹ is independently selected from halogen, hydroxyl, cyano, amino, nitro, C₁₋₆ alkylamino, di(C₁₋₆)alkylamino, C₃₋₆ cycloalkyl, aryl optionally substituted by 1 to 3 groups R³², heteroaryl optionally substituted by 1 to 3 groups R³², C₁₋₆ alkoxy, C₁₋₆ haloalkoxy, C₁₋₆ alkylthio, C₁₋₆ alkylcarbonyl or C₁₋₆ alkoxycarbonyl, each R²⁴ is independently halogen, hydroxyl, cyano, amino, nitro, C₁₋₆ alkylamino, di(C₁₋₆)alkylamino, C₁₋₆ alkyl, C₁₋₆ haloalkyl, C₁₋₆ alkoxy, C₁₋₆ haloalkoxy, C₁₋₆ alkoxy(C₁₋₆)alkoxy, carboxy, C₁₋₆ alkylthio, C₁₋₆ alkylcarbonyl C₁₋₆ alkoxycarbonyl or tri(C₁₋₄)alkylsilyl, each R³² is independently selected from halogen, hydroxyl, cyano, amino, nitro, C₁₋₆ alkylamino, di(C₁₋₆)alkylamino, C₁₋₆ alkyl, C₁₋₆ haloalkyl, C₃₋₆ cycloalkyl, C₁₋₆ alkoxy, C₁₋₆ haloalkoxy, C₁₋₆ alkylthio, C₁₋₆ alkylcarbonyl or C₁₋₆ alkoxycarbonyl and each R³³ is independently halogen, hydroxyl, cyano, amino, nitro, C₁₋₆ alkylamino, di(C₁₋₆)alkylamino, C₁₋₆ alkyl, C₁₋₆ haloalkyl, C₁₋₆ alkoxy, C₁₋₆ haloalkoxy, C₁₋₆ alkylthio, C₁₋₆ alkylcarbonyl or C₁₋₆ alkoxycarbonyl or two geminal groups R³³ form an oxo group.

10. A compound according to claim 9, wherein R² is C₁₋₄ alkyl optionally substituted by 1 to 4 groups R²⁵, C₁₋₄ haloalkyl optionally substituted by 1 to 4 groups R²⁵ or phenyl optionally substituted by 1 to 3 groups R⁴ and R³ is C₁₋₄ alkyl optionally substituted by 1 to 4 groups R²⁵, C₁₋₄ haloalkyl optionally substituted by 1 to 4 groups R²⁵, phenyl optionally substituted by 1 to 3 groups R⁴, C₁₋₄ alkoxy, or —NR⁷₂, wherein each R²⁵ is independently cyano, C₁₋₄ alkoxy, C₃₋₆ cycloalkyl, phenyl optionally substituted by 1-3 groups R⁴, heteroaryl optionally substituted by 1-3 groups R⁴, or C₁₋₄ alkoxycarbonyl.

11. A compound according to claim 1, wherein X is —NR⁵R⁶, R⁵ is hydrogen, C₁₋₄ alkyl optionally substituted by 1 to 4 groups R²⁴, C₁₋₄ haloalkyl optionally substituted by 1 to 4 groups R²⁴, C₃₋₆ cycloalkyl optionally substituted by 1 to 4 groups R²⁴, C₂₋₄ alkenyl, —SO₂R² or —C(O)R³ and R⁶ is hydrogen, C₁₋₄ alkyl optionally substituted by 1 to 4 groups R²⁴, C₁₋₄ haloalkyl optionally substituted by 1 to 4 groups R²⁴ or C₂₋₄ alkenyl or R⁵ and R⁶ together form a group ═C(R¹¹)NR⁷₂ and R² is C₁₋₄ alkyl, C₁₋₄ haloalkyl, or phenyl optionally substituted by 1 to 3 groups R⁴, R³ is C₁₋₄ alkyl optionally substituted by 1 to 4 groups R²⁵, C₁₋₄ haloalkyl optionally substituted by 1 to 4 groups R²⁵, phenyl optionally substituted by 1 to 3 groups R⁴, C₁₋₄ alkoxy, or —NR⁷₂, R¹¹ is hydrogen or C₁₋₄ alkyl and each R²⁴ is independently hydroxyl, cyano, C₁₋₄ alkoxy, C₁₋₄ alkoxy(C₁₋₄) alkoxy, carboxy, C₁₋₄ alkoxycarbonyl, or tri(C₁₋₄)alkylsilyl.

12. A compound according to claim 11, wherein X is —NR⁵R⁶ wherein R⁵ is hydrogen, C₁₋₄ alkyl optionally substituted with 1 or 2 hydroxy or C₁₋₄ alkoxy groups, C₁₋₄ haloalkyl optionally substituted with 1 or 2 hydroxy or C₁₋₄ alkoxy groups, C₃₋₆ cycloalkyl, C₂₋₄ alkenyl, —SO₂R², or —C(O)R³, wherein R² and R³ are each independently C₁₋₃ alkyl or phenyl and R⁶ is hydrogen, C₁₋₄ alkyl optionally substituted with 1 or 2 hydroxy or C₁₋₄ alkoxy groups, C₁₋₄ haloalkyl optionally substituted with 1 or 2 hydroxy or C₁₋₄ alkoxy groups, or C₂₋₄ alkenyl.

13. A compound according to claim 1, wherein Y is C₁₋₆ alkyl optionally substituted by 1 to 3 groups R³⁴, C₁₋₆ haloalkyl optionally substituted by 1 to 3 groups R³⁴, C₃₋₆ cycloalkyl optionally substituted by 1 to 3 groups R³⁵, C₂₋₆ alkenyl optionally substituted by 1 to 3 groups R³⁶ or C₂₋₆ alkynyl optionally substituted by 1 to 3 groups R³⁷ and each R³⁴ is independently halogen, cyano, nitro, hydroxyl, C₃₋₆ cycloalkyl, C₁₋₆ alkoxy, C₁₋₄ alkylthio, C₁₋₄ alkylcarbonyl, C₁₋₄ alkoxycarbonyl or two geminal groups R³⁴ form an oxo group, each R³⁵ is independently halogen, cyano, nitro, hydroxyl, C₁₋₆ alkyl, C₃₋₆ cycloalkyl, C₁₋₆ alkoxy, C₁₋₄ alkylthio, C₁₋₄ alkylcarbonyl or C₁₋₄ alkoxycarbonyl, each R³⁶ is independently halogen, cyano, nitro, C₃₋₆ cycloalkyl, C₁₋₆ alkoxy, C₁₋₄ alkylcarbonyl, C₁₋₄ alkoxycarbonyl or C₁₋₃alkylsulphonyl and each R³⁷ is independently halogen, cyano, C₃₋₆ cycloalkyl, C₁₋₄ alkylcarbonyl, C₁₋₄ alkoxycarbonyl or tri(C₁₋₃)alkylsilyl.

14. A compound according to claim 13, wherein Y is C₁₋₃ alkyl, C₁₋₃ haloalkyl, C₁₋₂ alkoxy(C₁₋₂)alkyl, cyclopropyl optionally substituted by 1 or 2 groups independently selected from halogen or C₁₋₆ alkyl, C₂₋₄ alkenyl, C₂₋₄ haloalkenyl or C₂₋₄ alkynyl optionally substituted by 1 or 2 groups independently selected from halogen or tri(C₁₋₃)alkylsilyl.

15. A compound according to claim 14, wherein Y is C₁₋₂ alkyl, C₁₋₂ haloalkyl, C₁₋₂ alkoxy(C₁₋₂)alkyl, C₂₋₄ alkenyl or C₂₋₄ alkynyl.

16. A compound according to claim 1, wherein Z is —C(O)R¹², C(S)R¹³, or —C(═NR¹⁴) R¹⁵ and R¹² is hydrogen, hydroxyl, C₁₋₂₀ alkoxy optionally substituted by 1 to 3 groups R³⁸, C₁₋₁₀ alkenyloxy optionally substituted by 1 to 3 groups R³⁸, C₃₋₆ cycloalkoxy optionally substituted by 1 to 3 groups R³⁸, C₁₋₁₀ alkylthio optionally substituted by 1 to 3 groups R³⁸, amino, C₁₋₆ alkylamino optionally substituted by 1 to 3 groups R³⁸ or di(C₁₋₆)alkylamino optionally substituted by 1 to 3 groups R³⁸, R¹³ is C₁₋₂₀ alkoxy optionally substituted by 1 to 3 groups R³⁸, C₃₋₆ cycloalkoxy optionally substituted by 1 to 3 groups R³⁸, C₁₋₁₀ alkylthio optionally substituted by 1 to 3 groups R³⁸, amino, C₁₋₆ alkylamino optionally substituted by 1 to 3 groups R³⁸ or di(C₁₋₆) alkylamino optionally substituted by 1 to 3 groups R³⁸, R¹⁴ is hydrogen, C₁₋₆ alkyl optionally substituted by 1 to 3 groups R³⁸, C₁₋₂₀ alkoxy optionally substituted by 1 to 3 groups R³⁸, C₃₋₆ cycloalkoxy optionally substituted by 1 to 3 groups R³⁸, amino, C₁₋₆ alkylamino optionally substituted by 1 to 3 groups R³⁸ or di(C₁₋₆)alkylamino optionally substituted by 1 to 3 groups R³⁸, R¹⁵ is hydrogen, C₁₋₂₀ alkoxy optionally substituted by 1 to 3 groups R³⁸, C₃₋₆ cycloalkoxy optionally substituted by 1 to 3 groups R³⁸, C₁₋₁₀ alkylthio optionally substituted by 1 to 3 groups R³⁸, amino, C₁₋₆ alkylamino optionally substituted by 1 to 3 groups R³⁸ or di(C₁₋₆) alkyamino optionally substituted by 1 to 3 groups R³⁸ and each R³⁸ is independently C₁₋₆ alkoxy, phenyl optionally substituted by 1 to 3 groups R³⁹ or heteroaryl optionally substituted by 1 to 3 groups R³⁹ and each R³⁹ is independently halogen, cyano, C₁₋₄ alkyl, C₁₋₄haloalkyl, C₁₋₃ alkoxy(C₁₋₃)alkyl, C₁₋₄ alkoxy, C₁₋₄ haloalkoxy, C₁₋₄ alkylsulphonyl, or C₁₋₄ alkoxycarbonyl.

17. A compound according to claim 16, wherein Z is —C(O)R¹².

18. A compound according to claim 15, wherein R¹² is hydroxyl, C₁₋₁₀ alkylthio, C₁₋₂₀ alkoxy optionally substituted by 1 or 2 groups R³⁸, C₁₋₁₀ alkenyloxy optionally substituted by 1 or 2 groups R³⁸ or C₁₋₂₀ haloalkoxy optionally substituted by 1 to 2 groups R³⁸.

19. A herbicidal composition comprising a compound as defined in claim 1, together with at least one agriculturally acceptable adjuvant or diluent.

20. A composition according to claim 19, which comprises a further herbicide in addition to the compound of formula (I).

21. A composition according to claim 19, which comprises a safener.

22. Use of a compound as defined in claim 1.

23. A method of controlling weeds in crops of useful plants, comprising applying to said weeds or claim 1.
